(19) United States
(12) Patent Application Publication
(10) Pub. No.: US 2009/0258342 A1
(43) Pub. Date: Oct. 15, 2009

(54) OPTIMIZED PROBES AND PRIMERS AND METHODS OF USING SAME FOR THE DETECTION, QUANTIFICATION AND GROUPING OF HIV-1

(75) Inventors: Matthew Ducar, Mattapan, MA (US); James R. Hully, Salisbury, MA (US); Julian Chandler, Allston, MA (US); William Lauzier, East Bridgewater, MA (US)

Correspondence Address: FOLEY & LARDNER LLP, 111 HUNTINGTON AVENUE, 26TH FLOOR, BOSTON, MA, 02199-7610, US

(73) Assignee: INTELLIGENT MEDICAL DEVICES, INC.

(21) Appl. No.: 12/418,352

(22) Filed: Apr. 3, 2009

Related U.S. Application Data
(60) Provisional application No. 61/043,609, filed on Apr. 9, 2008.

(51) Int. Cl. C12Q 1/70; C07H 21/04; C12P 19/34

(52) U.S. Cl. 435/5; 536/23.1; 435/91.2; 536/24.33; 536/24.3

(57) ABSTRACT

Described herein are optimized primers and probes useful for detecting, quantitating and grouping variant HIV-1 strains, and methods and kits of using the described primers and probes.

### RELATED APPLICATION

This application claims the benefit of U.S. Provisional Patent Application No. 61/043,609, filed Apr. 9, 2008, the contents of which are incorporated by reference herein in its entirety.

### BACKGROUND

HIV is a retrovirus that causes the acquired immunodeficiency syndrome (AIDS) in humans. It also has been called the human T-lymphotropic virus-III (HTLV-III), lymphadenopathy-associated virus (LAV), and AIDS-associated retrovirus (ARV). HIV has a very high genetic variability. This diversity is a result of a fast replication cycle, generating up to 10¹⁰ virions per day, coupled with a high mutation rate and the recombinogenic properties of the endogenous viral reverse transcriptase. Consequently, multiple variants of HIV reside in an infected patient. This variability leads to problems in detecting and targeting the virus, which is further compounded by the potential genetic recombination between co-infected variants leading to the emergence of circulating recombinant forms. Additionally, there are genetic pressures, such as drugs, that result in a continuous emergence of recombinant forms of HIV.

Two species of HIV infect humans: HIV-1 and HIV-2. HIV-1 is the cause of the majority of HIV infections globally. HIV-2 is less transmittable than HIV-1 and is largely confined to West Africa. Three groups of HIV-1 have been identified: M, N, and O. Group M is the most prevalent and is subdivided into at least eight subtypes (or clades).

Infection with HIV produces many clinical manifestations affecting almost every organ in the body, such as opportunistic infections, dermatologic disorders, vascular complications, neurological manifestations and myopathies. Examples of HIV-associated conditions or diseases include AIDS, Kaposi's sarcoma, Non-Hodgkins lymphoma, Pneumocystis carinii pneumonia, Pneumocystis jirovecii pneumonia, Candida esophagitis, Candida albicans infection, Pseudomonas aeruginosa infection, Staphylococcus aureus infection, Streptococcus pyogenes infection, Acinetobacter baumanni infection, Toxoplasma gondii infection, Toxoplasma encephalitis, Aspergillus infection, cryptosporidiosis, microsporidiosis, Cryptococcus neoformans infection, mycobacterium avium complex disseminated infection, Epstein-Barr virus infection, cytomegalovirus retinitis, progressive multifocal leukoencephalopathy from JC virus infection, HIV-associated dementia, central nervous system (CNS) malignancies, oral candidiasis, aseptic meningoencephalitis, disorders of the digestive tract, endocrine dysfunction, metabolic disorders, wasting syndrome, anemia, neutropenia, rheumatological syndromes, cervical cancer, anal cancer, rectal cancer, Burkitt's lymphoma, penicilliosis, tuberculosis, herpes virus 8 infection, herpes virus simplex 1 infection, human papillomavirus infection, cytomegalovirus infection, mycobacterial infection, rotavirus infection, adenovirus infection, astrovirus infection, esophagitis, chronic diarrhea due to Salmonella, Shigella, Listeria, or Campylobacter, or nephropathy.

Early detection, subsequent antiviral therapy and monitoring the effectiveness of the therapy by determining the viral load can have a significant impact on improved patient outcome. Several methods are currently available for detecting HIV-1, including cell culture, immunoassays, Western blotting and nucleic acid testing, e.g., real-time PCR, but such methods do not adequately address the broad genetic diversity of target HIV pathogens. A rapid and accurate diagnostic test for the detection, quantification and grouping of HIV-1, therefore, would support effective treatments and control of infection.

### SUMMARY

The present invention relates to nucleic acid probes and primers selective for detecting, quantitating and grouping viral genetic material from Human immunodeficiency virus (HIV) (Type 1)(HIV-1), but not genetic material from HIV (Type 2)(HIV-2), and methods and kits for using the probes and primers. The present invention is based on the identification of amplification primers and probes that are useful for generally detecting and/or quantitating the presence of HIV-1 in a sample, while also distinguishing the particular HIV-1 group, i.e., groups M, N, and O (referred to as a “grouping”).

In one embodiment, the present invention is directed to an isolated nucleic acid sequence comprising a nucleotide sequence selected from the group consisting of: SEQ ID NOS:1-23.

In another embodiment, the invention is directed to at least one forward and reverse primer pair for amplifying HIV-1 DNA selected from the group consisting of: (1) SEQ ID NOS: 1 and 2; (2) SEQ ID NOS: 1 and 19; (3) SEQ ID NOS: 1 and 20; (4) SEQ ID NOS: 1 and 21; (5) SEQ ID NOS: 1 and 22; (6) SEQ ID NOS: 3 and 2; (7) SEQ ID NOS: 3 and 19; (8) SEQ ID NOS: 3 and 20; (9) SEQ ID NOS: 3 and 21; (10) SEQ ID NOS: 3 and 22; (11) SEQ ID NOS: 15 and 2; (12) SEQ ID NOS: 15 and 19; (13) SEQ ID NOS: 15 and 20; (14) SEQ ID NOS: 15 and 21; (15) SEQ ID NOS: 15 and 22; (16) SEQ ID NOS: 16 and 2; (17) SEQ ID NOS: 16 and 19; (18) SEQ ID NOS: 16 and 20; (19) SEQ ID NOS: 16 and 21; (20) SEQ ID NOS: 16 and 22; (21) SEQ ID NOS: 17 and 2; (22) SEQ ID NOS: 17 and 19; (23) SEQ ID NOS: 17 and 20; (24) SEQ ID NOS: 17 and 21; (25) SEQ ID NOS: 17 and 22; (26) SEQ ID NOS: 18 and 2; (27) SEQ ID NOS: 18 and 19; (28) SEQ ID NOS: 18 and 20; (29) SEQ ID NOS: 18 and 21; and (30) SEQ ID NOS: 18 and 22.

In a particular embodiment, the present invention is directed to a primer comprising a sequence selected from the group consisting of: SEQ ID NOS: 2 and 19-22 for the production of cDNA from HIV-1 RNA.

In one embodiment, the present invention is directed to a set of oligonucleotides comprising at least one forward and reverse primer pair and one or more probes selected from the group consisting of sets 1-210 of Table 1.

In one embodiment, the present invention is directed to one or more probes, such as a probe triplex comprising three nucleic acid probes, that bind to a PCR product (amplicon) created by using the primer pair, wherein the probe(s) comprises a sequence selected from the group consisting of SEQ ID NOS:4-14 and 23. In a particular embodiment, the probe(s) is labeled. In one embodiment comprising a probe triplex, each of the three probe sequences is labeled with a different fluorogenic dye to detect and quantitate the three groups of HIV-1, e.g., Groups M, N and O. In a particular embodiment, the probe(s) comprises a fluorescent label, a chemiluminescent label, a radioactive label, a quenching molecule, biotin or gold. In a particular embodiment, the probe(s) is labeled for detecting and quantitating HIV-1. In a particular embodiment, the probe(s) is labeled for grouping HIV-1.

In one embodiment, the present invention is directed to a probe combination comprising three probes that bind to an amplified PCR product created by a primer pair, wherein the first probe comprises the sequence of SEQ ID NO:4 or SEQ ID NO:6, the second probe comprises the sequence of SEQ ID NOS:5, 7 or 23, and the third probe comprises a sequence selected from the group consisting of: SEQ ID NOS:8-14. Each probe is labeled with a different label for detecting a specific group of HIV-1.

In another embodiment, the present invention is directed to a method for detecting, quantitating and grouping HIV-1 RNA in a sample, comprising: a) contacting the HIV-1 RNA with a reverse transcriptase primer sequence selected from the group consisting of: SEQ ID NOS:2 and 19-22 for the production of cDNA; b) contacting the cDNA with at least one forward primer comprising the sequence selected from the group consisting of: SEQ ID NOS:1, 3 and 15-18 and at least one reverse primer sequence selected from the group consisting of: SEQ ID NOS:2 and 19-22 under conditions such that nucleic acid amplification occurs to yield an amplified PCR product; and c) contacting the amplified PCR product with one or more probes comprising one or more sequences selected from the group consisting of: SEQ ID NOS:4-14 and 23 under conditions such that binding occurs; wherein detection, quantification and grouping of an amplified PCR product by one or more probes is indicative of the presence of one or more of the groups of HIV-1 in the sample. Step c) is performed using a labeled probe(s) comprising a sequence that hybridizes to the amplicon generated by the forward and reverse primer pair group of step b).

In another embodiment, the invention is directed to a method for detecting, quantitating and grouping HIV-1 DNA in a sample, comprising: a) contacting the HIV-1 DNA with at least one forward primer comprising the sequence selected from the group consisting of: SEQ ID NOS:1, 3 and 15-18 and at least one reverse primer comprising the sequence selected from the group consisting of: SEQ ID NOS:2 and 19-22 under conditions such that nucleic acid amplification occurs to yield an amplified PCR product; and b) contacting the amplified PCR product with one or more probes, comprising one or more sequences selected from the group consisting of: SEQ ID NOS:4-14 and 23 under conditions such that hybridization or binding occurs; wherein detection, quantification and grouping of an amplified PCR product by one or more probes is indicative of the presence of one or more of the groups of HIV-1 in the sample. Step b) is performed using a labeled probe(s) comprising a sequence that hybridizes to the amplicon generated by the forward and reverse primer pair group of step a).

In both embodiments, the PCR amplicon is contacted by one or more probes comprising different sequences, wherein each sequence is indicative of a group of HIV-1. The one or more probes are fluorescently labeled and the step of detecting the binding of the probe to the amplified PCR product entails measuring the fluorescence of the sample. The probes comprising different sequences are labeled with different fluorescent labels.

In a particular embodiment, the amplified PCR product is contacted by two or more probes comprising different sequences, wherein each sequence is indicative of a group of HIV-1 (e.g., Groups M, N and O). In a particular embodiment, the one or more probes is fluorescently labeled and the step of detecting the binding of the probe(s) to the amplified sample entails measuring the fluorescence of the sample. The probe comprises a fluorescent reporter moiety and a quencher of fluorescence moiety. Upon probe hybridization with the amplified PCR product, the exonuclease activity of Taq Polymerase cleaves the probe into individual nucleotides some of which will contain the reporter and quencher, resulting in the unquenched emission of fluorescence, which is detected. An increase in the amplified PCR product causes a proportional increase in fluorescence, due to cleavage of the probe and release of the reporter moiety of the probe. The amplified PCR product is quantified in real time as it accumulates.

In a particular embodiment, three probes comprising different sequences are labeled with three different fluorescent labels. One probe comprises the sequence of SEQ ID NO: 4 or SEQ ID NO:6 (Group N probes), the second probe comprises a sequence selected from the group consisting of SEQ ID NOS: NOS: 5, 7 and 23 (group 0 probes) and the third probe comprises a sequence selected from the group consisting of SEQ ID NOS: 8-14 (Group M probes). Each probe is labeled with a unique fluorescent dye and detected in distinguishing channels for detection of Groups M, N or O of HIV-1. For example, the Group M probe is labeled with FAM dye and detected at 520 nm in one channel (e.g., channel A), the Group O probe is labeled with CAL-Fluor dye and detected at 590 nm in another channel (e.g., channel C), and the Group N probe is labeled with Quasar dye and detected at 670 nm in a different channel (e.g., channel E).

In one embodiment, the probes are molecular beacons. Molecular beacons are single-stranded probes that form a stem-and-loop structure. A fluorophore is covalently linked to one end of the stem and a quencher is covalently linked to the other end of the stem forming a stem hybrid. When a molecular beacon hybridizes to a target nucleic acid sequence, the probe undergoes a conformational change that results in the dissociation of the stem hybrid and, thus the fluorophore and the quencher move away from each other, enabling the probe to fluoresce brightly. Molecular beacons can be labeled with differently colored fluorophores to detect different target sequences. Any of the probes described herein may be designed and utilized as molecular beacons.

In an embodiment, the present invention is directed to a method for detecting, quantitating and grouping HIV-1 DNA in a sample comprising at least one primer pair and one or more probes selected from the group consisting of sets 1-210 of Table 1.

The sample is blood, serum, plasma, sputum, urine, stool, skin, cerebrospinal fluid, saliva, gastric secretions, semen, seminal fluid, breastmilk, tears, oropharyngeal swabs, nasopharyngeal swabs, throat swabs, nasal aspirates, nasal wash, fluids collected from the ear, eye, mouth, respiratory airways, spinal tissue or fluid, cerebral fluid, trigeminal ganglion sample, a sacral ganglion sample, adipose tissue, lymphoid tissue, placental tissue, upper reproductive tract tissue, gastrointestinal tract tissue, male genital tissue or fetal central nervous system tissue. The sample can be obtained from a mammal, e.g., a human.

Particular nucleic acids, methods and kits of the invention can be directed to particular combinations of primers and probes, e.g., those selected from the group consisting of SEQ ID NOS:1-23, especially the sets described in Table 1.

In one embodiment, the present invention is directed to a viral kit for detecting, quantitating and grouping an HIV-1 RNA sample, comprising: a) at least one reverse transcriptase primer comprising a sequence selected from the group consisting of: SEQ ID NOS:2 and 19-22 for cDNA production and nucleic acid amplification; b) at least one forward primer comprising the sequence selected from the group consisting of: SEQ ID NOS: 1, 3 and 15-18 for nucleic acid amplification; and c) one or more probes comprising a sequence selected from the group consisting of: SEQ ID NOS:4-14 and 23 for binding to an amplified nucleic acid product.

In another embodiment, the present invention is directed to a proviral kit for detecting, quantitating and grouping an HIV-1 DNA sample, comprising: a) at least one forward primer comprising the sequence selected from the group consisting of: SEQ ID NOS:1, 3 and 15-18 for nucleic acid amplification; b) at least one reverse primer comprising the sequence selected from the group consisting of: SEQ ID NO:2, and 19-22 for nucleic acid amplification; and c) one or more probes comprising a sequence selected from the group consisting of: SEQ ID NOS: 4-14 and 23 for binding to an amplified nucleic acid product.

In a particular embodiment, the one or more probes in the viral and proviral kits comprise different sequences, wherein each sequence is indicative of a group of HIV-1 and are fluorescently labeled. The probes may comprise different sequences, which are labeled with different fluorescent labels. The viral and proviral kits may include at least one primer pair and one or more probes selected from the group consisting of sets 1-210 of Table 1.

In an additional embodiment, the present invention is directed to a kit for detecting and quantitating a targeted HIV sequence derived from an artificial construct, such as a plasmid, comprising: a) at least one forward primer comprising the sequence selected from the group consisting of SEQ ID NOS:1, 3 and 15-18; b) at least one reverse primer comprising the sequence selected from the group consisting of SEQ ID NOS:2 and 19-22; and c) one or more probes comprising a sequence selected from the group consisting of SEQ ID NOS:4-14 and 23.

In one embodiment, the present invention is directed to A method for detecting, quantitating and grouping an HIV-1-associated condition or disease comprising: (1) contacting at least one forward and reverse primer pair selected from the group consisting of: SEQ ID NOS:1-3 and 15-22 to a sample; (2) conducting a reverse transcriptase-polymerase chain reaction, a polymerase chain reaction or signal amplification; and (3) detecting and grouping the generation of an amplified product using one or more probes selected from the group consisting of: SEQ ID NOS:4-14 and 23; wherein the generation of an amplified product indicates the presence of HIV-1 virus in the sample.

In a particular embodiment, the HIV-1-associated condition or disease is an HIV-1 infection that is identifiable and present in a sample selected from the group consisting of: blood, serum, plasma, sputum, urine, stool, skin, cerebrospinal fluid, saliva, gastric secretions, semen, seminal fluid, breastmilk, tears, oropharyngeal swabs, nasopharyngeal swabs, throat swabs, nasal aspirates, nasal wash, fluids collected from the ear, eye, mouth, respiratory airways, spinal tissue or fluid, cerebral fluid, trigeminal ganglion sample, a sacral ganglion sample, adipose tissue, lymphoid tissue, placental tissue, upper reproductive tract tissue, gastrointestinal tract tissue, male genital tissue and fetal central nervous system tissue. In a particular embodiment, the sample is a human sample. In a particular embodiment, the HIV-1-associated condition or disease is AIDS, Kaposi's sarcoma, Non-Hodgkins lymphoma, Pneumocystis carinii pneumonia, Pneumocystis jirovecii pneumonia, Candida esophagitis, Candida albicans infection, Pseudomonas aeruginosa infection, Staphylococcus aureus infection, Streptococcus pyogenes infection, Acinetobacter baumanni infection, Toxoplasma gondii infection, Toxoplasma encephalitis, Aspergillus infection, cryptosporidiosis, microsporidiosis, Cryptococcus neoformans infection, mycobacterium avium complex disseminated infection, Epstein-Barr virus infection, cytomegalovirus retinitis, progressive multifocal leukoencephalopathy from JC virus infection, HIV-associated dementia, central nervous system (CNS) malignancies, oral candidiasis, aseptic meningoencephalitis, disorders of the digestive tract, endocrine dysfunction, metabolic disorders, wasting syndrome, anemia, neutropenia, rheumatological syndromes, cervical cancer, anal cancer, rectal cancer, Burkitt's lymphoma, penicilliosis, tuberculosis, herpes virus 8 infection, herpes virus simplex 1 infection, human papillomavirus infection, cytomegalovirus infection, mycobacterial infection, rotavirus infection, adenovirus infection, astrovirus infection, esophagitis, chronic diarrhea due to Salmonella, Shigella, Listeria, or Campylobacter, or nephropathy.

In one embodiment, the present invention is directed to an oligonucleotide comprising a nucleotide sequence selected from the group consisting of: SEQ ID NOS: 1-23 for isolating nucleic acid sequences derived from any HIV-1 genome or artificial construct of an HIV-1 genome containing the target sequence.

In a particular embodiment, the present invention is directed to an oligonucleotide comprising a nucleotide sequence selected from the group consisting of: SEQ ID NOS:1-23 for sequencing nucleic acid sequences derived from any HIV-1 genome or artificial construct of the HIV-1 genome containing the target sequence.

In another embodiment, the present invention is directed to an isolated nucleic acid sequence comprising a sequence selected from the group consisting of: SEQ ID NOS:1-3 and 15-22, wherein the sequence is used as a probe to detect an HIV-1 viral variant.

In a particular embodiment, the present invention is directed to an oligonucleotide comprising a nucleotide sequence selected from the group consisting of: SEQ ID NOS:1-23, in which the oligonucleotide does not cross react with any of the following organisms: Adenovirus types 2, 3 and 7; BK human polyoma virus; CMV; EBV; HHV-6 A; HHV-6 B; HHV-7; HHV-8; HIV-2; HSV-2; HSV-2; HTLV-I; HTLV-II; human hepatitis A virus; human hepatitis B virus; hepatitis C virus; JC virus; human papillomavirus types 16; human papillomavirus types 18; VZV; Candida albicans; Escherichia coli; Klebsiella pneumoniae; Morganella morganii; Proteus mirabilis; Pseudomonas aeruginosa; Serratia marcescens; Staphylococcus aureus; Staphylococcus haemolyticus; Staphylococcus hominis; Staphylococcus sciuri; Staphylococcus simulans; Staphylococcus epidermidis; and Staphylococcus intermedius.

The term “cross react” means the oligonucleotide does not bind with sufficient efficiency to allow the detection of a signal utilizing standard polymerase chain reaction conditions known to one of skill in the art. Substantially no cross-reactivity refers to a degree of cross-reactivity that allows for the identification of a specific target reactivity over reactivity with a cross-reactive target and/or background.

### DETAILED DESCRIPTION

Described herein are nucleic acid primers and probes for detecting, quantitating and grouping viral genetic material, especially HIV viruses, including HIV-1 (Type 1) and the groups and subtypes of Type 1, namely HIV-1 groups “M,” “N,” and “O,” as well as the at least eight different clades or subtypes of group M, but not HIV-2 (Type 2). Also described herein are methods for designing and optimizing the respective primer and probe sequences that are useful for specifically detecting, quantitating and grouping these HIV-1 viruses in a given sample. Of particular interest in this regard is the ability of the disclosed primers and probes—as well as those that can be designed according to the disclosed methods—to specifically detect, quantitate and group strains and variants of HIV-1 variant-specific genomic mutations. The optimized primers and probes are useful, therefore, for identifying, quantitating, and grouping the causative or contributing agents of HIV-1 infection, whereupon an appropriate treatment can then be administered to the individual, resulting in steps performed to eradicate HIV-1.

The term “HIV” is used in its inclusive sense herein to encompass HIV Type 1 including the three HIV-1 groups, M (and its at least eight clades (subtypes)), N and O; and HIV-2). The term “HIV-1” is also used in its inclusive sense to encompass the three groups, M, N and O, as well as the eight M clades or subtypes. The compositions and methods described herein are useful for the detection, quantitation and grouping of one or more of (1) HIV-1, (2) HIV-1M (any of the at least 8 clades or subtypes), (3) HIV-1N, and (4) HIV-10; but not HIV-2.

Described herein is a robust bioinformatic analytical system that is useful for performing a comprehensive analysis of all known target HIV-1 sequences to design primers and probes with the best possible sensitivity and specificity. That is, the primers and probes are useful for detecting, quantitating and grouping HIV-1 in a singleplex or multiplex format.

The present invention provides one or more pairs of PCR primers that can anneal to HIV-1 variants and thereby amplify a PCR product from a biological sample. The present invention provides at least a first PCR primer and a second PCR primer, each of which comprises a nucleotide sequence designed according to the inventive principles disclosed herein, which are used together to positively identify the presence of HIV-1 in a sample regardless of the actual nucleotide composition of the infecting HIV-1 variant(s). The generation of an amplified PCR product or products from a sample using the primer pairs disclosed herein is diagnostically indicative of a variant of HIV being present in the sampled material and at least diagnostically indicative of the presence of an HIV-1 infection. Of note, each of the primer sequences can be used as a probe to detect and/or quantitate HIV-1 viral variants.

Also provided by the present invention are probes that hybridize to amplified PCR products or unamplified sample sequences. A probe can be labeled, for example, such that when it binds to an internal PCR product target sequence, or after it has been cleaved after binding, a fluorescent signal is emitted that is detectable under various spectroscopy and light-measurement apparatuses. The use of a labeled probe, therefore, can enhance the specificity of the PCR-based amplification of variant HIV DNA because it permits the detection and/or quantification of virus DNA at low template concentrations that might not be conducive to visual detection, such as detection of a gel-stained PCR product.

Primers and probes are nucleic acid sequences that anneal to a viral genomic sequence, e.g., HIV-1 (the “target”). The target sequence can be, for example, a viral genome or a subset, “region”, of a viral genome. In one embodiment, the entire genomic sequence can be “scanned” for optimized primers and probes useful for detecting and/or quantitating viral variants. In other embodiments, particular regions of the viral genome can be scanned, e.g., regions that are documented in the literature as being useful for detecting multiple variants, regions that are conserved, or regions where sufficient information is available in, for example, a public database, with respect to viral variants.

The target HIV-1 sequences utilized in this invention are genomic sequences. According to the methods of this invention, a consensus is not generated from the alignment of these genomic HIV-1 sequences. Each of the target HIV-1 genomic sequences is analyzed independently. The methods of the invention utilize an alignment of the target HIV-1 sequences to generate candidate primer and probe sequences. The set of all possible primers and probes can include, for example, sequences that include the variability at every site based on the known viral variants. The primers and probes are generated such that the primers and probes are able to anneal to a particular variant under high stringency conditions. For example, one of skill in the art recognizes that for any particular sequence, it is possible to provide more than one oligonucleotide sequence that will anneal to the particular target sequence, even under high stringency conditions. The set of primers and probes to be sampled for the purposes of the present invention includes, for example, all such oligonucleotides for all viral variant sequences.

Stringent hybridization conditions include a solution comprising about 1 M Na⁺ at 25° C. to 30° C. below the Tm; e.g., 5×SSPE, 0.5% SDS, at 65° C.; see, Ausubel, et al., Current Protocols in Molecular Biology, Greene Publishing, 1995; Sambrook et al., Molecular Cloning: A Laboratory Manual, Cold Spring Harbor Press, 1989). Tm is dependent on both the G+C content and the concentration of salt ions, e.g., Na⁺ and K⁺. A formula to calculate the Tm of nucleic acid molecules greater than about 500 bp is Tm=81.5+0.41(% (G+C))−log₁₀[Na⁺]. Washing conditions are generally performed at least at equivalent stringency conditions as the hybridization. If the background levels are high, washing can be performed at higher stringency, such as around 15° C. below the Tm.

The term “Tm” means the temperature at which a population of double-stranded nucleic acid molecules becomes half-dissociated into single strands. Methods for calculating the Tm of nucleic acids are well known in the art (Berger and Kimmel (1987) Meth. Enzymol., Vol. 152: Guide To Molecular Cloning Techniques, San Diego: Academic Press, Inc. and Sambrook et al. (1989) Molecular Cloning: A Laboratory Manual, (2nd ed.) Vols. 1-3, Cold Spring Harbor Laboratory).

The methods of the invention provide optimal primer and probe sequences because they hybridize to all or a subset of HIV-1 variants. Once optimized oligonucleotides are identified that can anneal to viral variants, the sequences can then further be optimized for use, for example, in conjunction with another optimized sequence as a “primer pair” or for use as a probe.

In other embodiments of the invention, the methods of determining the primers and probes further comprise the step of evaluating which target nucleic acid variant sequences are hybridized by an optimal forward primer and an optimal reverse primer, for example, by determining the number of base differences between target nucleic acid variant sequences in a database. For example, the evaluating step can comprise performing an in silico polymerase chain reaction, involving (1); rejecting the forward primer and/or reverse primer if it does not amplify a medically valuable nucleic acid; and/or (2) determining the secondary structure of the forward primer, reverse primer, and/or target. In one embodiment, the evaluating step includes evaluating whether the forward primer sequence, reverse primer sequence, and/or probe sequence hybridizes to sequences in the database other than the nucleic acid sequences that are representative of the target variants.

The present invention provides sets of oligonucleotides. Each set may contain at least one forward primer, at least one reverse primer, and one or more probes. The present invention also provides extended forward primers, reverse primers and probes.

The present invention provides oligonucleotides that have preferred primer and probe qualities. These qualities are specific to the sequences of the optimized primers and probes, however, one of skill in the art would recognize that other molecules with similar sequences could also be used. The oligonucleotides provided herein comprise a sequence that shares at least about 60-70% identity with a sequence identified as SEQ ID NOS: 1 through 23. In addition, the sequences can be incorporated into longer sequences, provided they function to specifically anneal to and identify viral variants. In another embodiment, the invention provides a nucleic acid comprising a sequence that shares at least about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity with the sequences disclosed herein or complement thereof.

The terms “homology” or “identity” or “similarity” refer to sequence relationships between two nucleic acid molecules and can be determined by comparing a nucleotide position in each sequence when aligned for purposes of comparison. The term “homology” refers to the relatedness of two nucleic acid or protein sequences. The term “identity” refers to the degree to which nucleic acids are the same between two sequences. The term “similarity” refers to the degree to which nucleic acids are the same, but includes neutral degenerate nucleotides that can be substituted within a codon without changing the amino acid identity of the codon, as is well known in the art. The primer and/or probe nucleic acid sequences of the invention are complementary to the target nucleic acid sequence. The primer and/or probe nucleic acid sequences of the invention are optimal for identifying numerous variants of a target nucleic acid, e.g., from an HIV-1 pathogen. In an embodiment, the nucleic acids of the invention are primers for the synthesis (e.g., amplification) of target nucleic acid variants and/or probes for identification, isolation, detection, quantification, grouping or analysis of target nucleic acid variants, e.g., an amplified target nucleic acid variant that is amplified using the primers of the invention.

The present oligonucleotides hybridize with more than one viral variant (variants as determined by differences in their genomic sequence). The probes and primers provided herein can, for example, allow for the detection, quantification and grouping of currently identified HIV-1 viral variants or a subset thereof. In addition, the primers and probes of the present invention, depending on the variant sequence(s), can allow for the detection, quantification and grouping of previously unidentified HIV-1 viral variants. The methods of the invention provide for optimal primers and probes, and sets thereof, and combinations of sets thereof, which can hybridize with a larger number of target variants than available primers and probes.

In other aspects, the invention also provides vectors (e.g., plasmid, phage, expression), cell lines (e.g., mammalian, insect, yeast, bacterial), and kits comprising any of the sequences of the invention described herein. The invention further provides target nucleic acid variant sequences that are identified, for example, using the methods of the invention. In an embodiment, the target nucleic acid variant sequence is an amplification product. In another embodiment, the target nucleic acid variant sequence is a native or synthetic nucleic acid. The primers, probes, and target nucleic acid variant sequences, vectors, cell lines, and kits can have any number of uses, such as diagnostic, investigative, confirmatory, monitoring, predictive or prognostic.

A diagnostic kit is provided by the present invention that comprises one or more of the oligonucleotides described herein, which are useful for detecting, quantitating and/or grouping HIV-1 infection in an individual and/or from a sample. An individual can be a human male, human female, human adult, human child, or human fetus. A sample includes any item, surface, material, clothing, or environment in which it may be desirable to test for the presence of HIV-1 variants. Thus, for instance, the present invention includes testing door handles, faucets, table surfaces, elevator buttons, chairs, toilet seats, sinks, kitchen surfaces, children's cribs, bed linen, pillows, keyboards, and so on, for the presence of HIV-1 variants.

A probe of the present invention can comprise a label, such as a fluorescent label, a chemiluminescent label, a radioactive label, biotin, gold, dendrimers, aptamer, enzymes, proteins, quenchers and molecular motors. In an embodiment, the label is a fluorescent label, such as, for example, FAM dye, CAL-Fluor Red and Quasar dye. In an embodiment, the probe is a hydrolysis probe, such as, for example, a TaqMan® probe. In other embodiments, the probes of the invention are molecular beacons, SYBR Green® primers, or fluorescence energy transfer (FRET) probes.

Oligonucleotides not only include primers that are useful for conducting the aforementioned PCR amplification reactions, but also include oligonucleotides that are attached to a solid support, such as, for example, a microarray, multiwell plate, column, bead, glass slide, polymeric membrane, glass microfiber, plastic tubes, cellulose or carbon nanostructures. Hence, detection, quantification and grouping of HIV-1 variants can be performed by exposing such an oligonucleotide-covered surface to a sample such that the binding of a complementary variant DNA sequence to a surface-attached oligonucleotide elicits a detectable signal or reaction. Oligonucleotides also include primers for isolating and sequencing nucleic acid sequences derived from any identified or yet to be isolated and identified HIV-1 genome.

One embodiment uses solid support-based oligonucleotide hybridization methods to detect gene expression. Solid support-based methods suitable for practicing the present invention are widely known and are described (PCT application WO 95/11755; Huber et al., Anal. Biochem., 299:24, 2001; Meiyanto et al., Biotechniques, 31:406, 2001; Relogio et al., Nucleic Acids Res., 30:e51, 2002; the contents of which are incorporated herein by reference in their entirety). Any solid surface to which oligonucleotides can be bound, covalently or non-covalently, can be used. Such solid supports include, but are not limited to, filters, polyvinyl chloride dishes, silicon or glass based chips.

In certain embodiments, the nucleic acid molecule can be directly bound to the solid support or bound through a linker arm, which is typically positioned between the nucleic acid sequence and the solid support. A linker arm that increases the distance between the nucleic acid molecule and the substrate can increase hybridization efficiency. There are a number of ways to position a linker arm. In one approach, the solid support is coated with a polymeric layer that provides linker arms with a plurality of reactive ends/sites. A common example of this type is glass slides coated with poly-1-lysine (U.S. Pat. No. 5,667,976, the contents of which are incorporated herein by reference in its entirety), which are commercially available. Alternatively, the linker arm can be synthesized as part of or conjugated to the nucleic acid molecule, and then this complex is bonded to the solid support. One approach, for example, takes advantage of the extremely high affinity biotin-streptavidin interaction. The streptavidin-biotinylated reaction is stable enough to withstand stringent washing conditions and is sufficiently stable that it is not cleaved by laser pulses used in some detection systems, such as matrix-assisted laser desorption/ionization time of flight (MALDI-TOF) mass spectrometry. Therefore, streptavidin can be covalently attached to a solid support, and a biotinylated nucleic acid molecule will bind to the streptavidin-coated surface. In one version of this method, an amino-coated silicon wafer is reacted with the n-hydroxysuccinimido-ester of biotin and complexed with streptavidin. Biotinylated oligonucleotides are bound to the surface at a concentration of about 20 fmol DNA per mm².

Alternatively, one can directly bind DNA to the support using carbodiimides, for example. In one such method, the support is coated with hydrazide groups, then treated with carbodiimide. Carboxy-modified nucleic acid molecules are then coupled to the treated support. Epoxide-based chemistries are also being employed with amine modified oligonucleotides. Other chemistries for coupling nucleic acid molecules to solid substrates are known to those of skill in the art.

The nucleic acid molecules, e.g., the primers and probes of the present invention, must be delivered to the substrate material, which is suspected of containing or is being tested for the presence, quantity and type of HIV-1 molecules. Because of the miniaturization of the arrays, delivery techniques must be capable of positioning very small amounts of liquids in very small regions, very close to one another and amenable to automation. Several techniques and devices are available to achieve such delivery. Among these are mechanical mechanisms (e.g., arrayers from GeneticMicroSystems, MA, USA) and ink-jet technology. Very fine pipets can also be used.

Other formats are also suitable within the context of this invention. For example, a 96-well format with fixation of the nucleic acids to a nitrocellulose or nylon membrane may also be employed.

After the nucleic acid molecules have been bound to the solid support, it is often useful to block reactive sites on the solid support that are not consumed in binding to the nucleic acid molecule. In the absence of the blocking step, excess primers and/or probes can, to some extent, bind directly to the solid support itself, giving rise to non-specific binding. Non-specific binding can sometimes hinder the ability to detect low levels of specific binding. A variety of effective blocking agents (e.g., milk powder, serum albumin or other proteins with free amine groups, polyvinylpyrrolidine) can be used and others are known to those skilled in the art (U.S. Pat. No. 5,994,065, the contents of which are incorporated herein by reference in their entirety). The choice depends at least in part upon the binding chemistry.

One embodiment uses oligonucleotide arrays, e.g., microarrays, that can be used to simultaneously observe the expression of a number of HIV-1 variant genes, such as the envelope protein gene (env). Oligonucleotide arrays comprise two or more oligonucleotide probes provided on a solid support, wherein each probe occupies a unique location on the support. The location of each probe may be predetermined, such that detection of a detectable signal at a given location is indicative of hybridization to an oligonucleotide probe of a known identity. Each predetermined location can contain more than one molecule of a probe, but each molecule within the predetermined location has an identical sequence. Such predetermined locations are termed features. There can be, for example, from 2, 5, 10, 100, 1,000, 2,000 or 5,000 or more of such features on a single solid support. In one embodiment, each oligonucleotide is located at a unique position on an array at least 2, at least 3, at least 4, at least 5, at least 6, or at least 10 times.

Oligonucleotide probe arrays for detecting gene expression can be made and used according to conventional techniques described (Lockhart et al., Nat. Biotech., 14:1675-1680, 1996; McGall et al., Proc. Natl. Acad. Sci. USA, 93:13555, 1996; Hughes et al., Nat. Biotechnol., 19:342, 2001). A variety of oligonucleotide array designs are suitable for the practice of this invention.

A detectable molecule, also referred to herein as a label, generally can be incorporated or added to an array's probe nucleic acid sequences. Many types of molecules can be used within the context of this invention. Such molecules include, but are not limited to, fluorochromes, chemiluminescent molecules, chromogenic molecules, radioactive molecules, mass spectrometry tags, proteins, and the like. Other labels will be readily apparent to one skilled in the art.

Oligonucleotide probes used in the methods of the present invention, including microarray techniques, can be generated using PCR. PCR primers used in generating the probes are chosen, for example, based on the sequences of SEQ ID NOS: 1-23. In one embodiment, oligonucleotide control probes also are used. Exemplary control probes can fall into at least one of three categories referred to herein as (1) normalization controls, (2) expression level controls, (3) process controls and (4) negative controls. In microarray methods, one or more of these control probes can be provided on the array with the inventive cell cycle gene-related oligonucleotides.

Normalization controls correct for dye biases, tissue biases, dust, slide irregularities, malformed slide spots, etc. Normalization controls are oligonucleotide or other nucleic acid probes that are complementary to labeled reference oligonucleotides or other nucleic acid sequences that are added to the nucleic acid sample to be screened. The signals obtained from the normalization controls, after hybridization, provide a control for variations in hybridization conditions, label intensity, reading efficiency and other factors that can cause the signal of a perfect hybridization to vary between arrays. The normalization controls also allow for the semi-quantification of the signals from other features on the microarray. In one embodiment, signals (e.g., fluorescence intensity or radioactivity) read from all other probes used in the method are divided by the signal from the control probes, thereby normalizing the measurements.

Virtually any probe can serve as a normalization control. Hybridization efficiency varies, however, with base composition and probe length. Preferred normalization probes are selected to reflect the average length of the other probes being used, but they also can be selected to cover a range of lengths. Further, the normalization control(s) can be selected to reflect the average base composition of the other probe(s) being used. In one embodiment, only one or a few normalization probes are used, and they are selected such that they hybridize well (i.e., without forming secondary structures) and do not match any test probes. In one embodiment, the normalization controls are mammalian genes.

“Negative control” probes are not complementary to any of the test oligonucleotides (i.e., the inventive cell cycle gene-related oligonucleotides), normalization controls, or expression controls. In one embodiment, the negative control is a mammalian gene that is not complementary to any other sequence in the sample.

The terms “background” and “background signal intensity” refer to hybridization signals resulting from non-specific binding or other interactions between the labeled target nucleic acids (e.g., mRNA present in the biological sample) and components of the oligonucleotide array. Background signals also can be produced by intrinsic fluorescence of the array components themselves. A single background signal can be calculated for the entire array, or a different background signal can be calculated for each target nucleic acid. In a one embodiment, background is calculated as the average hybridization signal intensity for the lowest five to ten percent of the oligonucleotide probes being used, or, where a different background signal is calculated for each target gene, for the lowest five to ten percent of the probes for each gene. Where the oligonucleotide probes corresponding to a particular HIV-1 target hybridize well and, hence, appear to bind specifically to a target sequence, they should not be used in a background signal calculation. Alternatively, background can be calculated as the average hybridization signal intensity produced by hybridization to probes that are not complementary to any sequence found in the sample (e.g., probes directed to nucleic acids of the opposite sense or to genes not found in the sample). In microarray methods, background can be calculated as the average signal intensity produced by regions of the array that lack any oligonucleotides probes at all.

In an alternative embodiment, the nucleic acid molecules are directly or indirectly coupled to an enzyme. Following hybridization, a chromogenic substrate is applied and the colored product is detected by a camera, such as a charge-coupled camera. Examples of such enzymes include alkaline phosphatase, horseradish peroxidase and the like. The invention also provides methods of labeling nucleic acid molecules with cleavable mass spectrometry tags (CMST; U.S. Patent No. 60/279,890). After an assay is complete, and the uniquely CMST-labeled probes are distributed across the array, a laser beam is sequentially directed to each member of the array. The light from the laser beam both cleaves the unique tag from the tag-nucleic acid molecule conjugate and volatilizes it. The volatilized tag is directed into a mass spectrometer. Based on the mass spectrum of the tag and knowledge of how the tagged nucleotides were prepared, one can unambiguously identify the nucleic acid molecules to which the tag was attached (WO 9905319, the entire contents of which are hereby incorporated by reference).

The nucleic acids, primers and probes of the present invention can be labeled readily by any of a variety of techniques. When the diversity panel is generated by amplification, the nucleic acids can be labeled during the reaction by incorporation of a labeled dNTP or use of labeled amplification primer. If the amplification primers include a promoter for an RNA polymerase, a post-reaction labeling can be achieved by synthesizing RNA in the presence of labeled NTPs. Amplified fragments that were unlabeled during amplification or unamplified nucleic acid molecules can be labeled by one of a number of end labeling techniques or by a transcription method, such as nick-translation, random-primed DNA synthesis. Details of these methods are known to one of skill in the art and are set out in methodology books. Other types of labeling reactions are performed by denaturation of the nucleic acid molecules in the presence of a DNA-binding molecule, such as RecA, and subsequent hybridization under conditions that favor the formation of a stable RecA-incorporated DNA complex.

In another embodiment, PCR-based methods are used to detect gene expression. These methods include reverse-transcriptase-mediated polymerase chain reaction (RT-PCR) including real-time and endpoint quantitative reverse-transcriptase-mediated polymerase chain reaction (Q-RTPCR). These methods are well known in the art. For example, methods of quantitative PCR can be carried out using kits and methods that are commercially available from, for example, Applied BioSystems and Stratagene®. See also Kochanowski, Quantitative PCR Protocols (Humana Press, 1999); Innis et al., supra.; Vandesompele et al., Genome Biol., 3:RESEARCH0034, 2002; Stein, Cell Mol. Life Sci. 59:1235, 2002.

The forward and reverse amplification primers and internal hybridization probe is designed to hybridize specifically and uniquely with one nucleotide derived from the transcript of a target gene. In one embodiment, the selection criteria for primer and probe sequences incorporates constraints regarding nucleotide content and size to accommodate TaqMan®requirements. In another embodiment, the selection criteria for primer and probe sequences incorporates constraints regarding nucleotide content and size to accommodate molecular probe requirements. SYBR Green® can be used as a probe-less Q-RTPCR alternative to the TaqMan®-type assay, discussed above (ABI Prism® 7900 Sequence Detection System User Guide Applied Biosystems, chap. 1-8, App. A-F. (2002)). A device measures changes in fluorescence emission intensity during PCR amplification. The measurement is done in “real time,” that is, as the amplification product accumulates in the reaction. Other methods can be used to measure changes in fluorescence resulting from probe digestion. For example, fluorescence polarization can distinguish between large and small molecules based on molecular tumbling (U.S. Pat. No. 5,593,867, the entire contents of which are hereby incorporated by reference).

The primers and probes of the present invention may anneal to or hybridize to various HIV-1 genetic material or genetic material derived therefrom, such as RNA, DNA, cDNA, or a PCR product.

A “sample” that is tested for the presence of an HIV-1 variant includes, but is not limited to, for example, blood, serum, plasma, sputum, urine, stool, skin, cerebrospinal fluid, saliva, gastric secretions, semen, seminal fluid, breast milk, and tears. A sample can be obtained by an oropharyngeal swab, nasopharyngeal swab, throat swab, nasal aspirate, nasal wash, fluid collected from the ear, eye, mouth, or respiratory airway, spinal tissue or fluid, cerebral fluid, trigeminal ganglion sample, a sacral ganglion sample, adipose tissue, lymphoid tissue, placental tissue, upper reproductive tract tissue, gastrointestinal tract tissue, male genital tissue and fetal central nervous system tissue. The tissue sample may be fresh, fixed, preserved, or frozen. A sample also includes any item, surface, material, or clothing, or environment in which it may be desirable to test for the presence of HIV-1 variant(s). Thus, for instance, the present invention includes testing door handles, faucets, table surfaces, elevator buttons, chairs, toilet seats, sinks, kitchen surfaces, children's cribs, bed linen, pillows, keyboards, and so on, for the presence of HIV-1 variants.

The present invention utilizes primers and probes in a method for detecting, quantitating and grouping an HIV-1-associated condition or disease, wherein the HIV-1-associated condition or disease is AIDS, Kaposi's sarcoma, Non-Hodgkins lymphoma, Pneumocystis carinii pneumonia, Pneumocystis jirovecii pneumonia, Candida esophagitis, Candida albicans infection, Pseudomonas aeruginosa infection, Staphylococcus aureus infection, Streptococcus pyogenes infection, Acinetobacter baumanni infection, Toxoplasma gondii infection, Toxoplasma encephalitis, Aspergillus infection, cryptosporidiosis, microsporidiosis, Cryptococcus neoformans infection, mycobacterium avium complex disseminated infection, Epstein-Barr virus infection, cytomegalovirus retinitis, progressive multifocal leukoencephalopathy from JC virus infection, HIV-associated dementia, central nervous system (CNS) malignancies, oral candidiasis, aseptic meningoencephalitis, disorders of the digestive tract, endocrine dysfunction, metabolic disorders, wasting syndrome, anemia, neutropenia, rheumatological syndromes, cervical cancer, anal cancer, rectal cancer, Burkitt's lymphoma, penicilliosis, tuberculosis, herpes virus 8 infection, herpes virus simplex 1 infection, human papillomavirus infection, cytomegalovirus infection, mycobacterial infection, rotavirus infection, adenovirus infection, astrovirus infection, esophagitis, chronic diarrhea due to Salmonella, Shigella, Listeria, or Campylobacter, or nephropathy.

The target nucleic acid variant that is amplified may be RNA or DNA or a modification thereof. If the target nucleic acid variant is RNA, the RNA can be reverse transcribed into cDNA using a reverse transcriptase primer and reverse transcriptase. The amplifying step may comprise an isothermal or non-isothermal reaction such as polymerase chain reaction, Scorpion® primers, molecular beacons, SimpleProbes®, HyBeacons®, cycling probe technology, Invader Assay, self-sustained sequence replication, nucleic acid sequence-based amplification, ramification amplifying method, hybridization signal amplification method, rolling circle amplification, multiple displacement amplification, thermophilic strand displacement amplification, transcription-mediated amplification, ligase chain reaction, signal mediated amplification of RNA, split promoter amplification, Q-Beta replicase, isothermal chain reaction, one cut event amplification, loop-mediated isothermal amplification, molecular inversion probes, ampliprobe, headloop DNA amplification, and ligation activated transcription. The amplifying step can be conducted on a solid support, such as a multiwell plate, array, column, bead, glass slide, polymeric membrane, glass microfiber, plastic tubes, cellulose, and carbon nanostructures. The amplifying step also comprises in situ hybridization. The detecting step can comprise gel electrophoresis, fluorescence resonant energy transfer, or hybridization to a labeled probe, such as a probe labeled with biotin, at least one fluorescent moiety, an antigen, a molecular weight tag, and a modifier of probe Tm. The detecting step can also comprise the incorporation of a label (e.g. fluorescent, mass or radioactive) during an extension reaction. The detecting step comprises measuring fluorescence, mass, charge, and/or chemiluminescence.

Hybridization may be detected in a variety of ways and with a variety of equipment. In general, the methods can be categorized as those that rely upon detectable molecules incorporated into the diversity panels and those that rely upon measurable properties of double-stranded nucleic acids (e.g., hybridized nucleic acids) that distinguish them from single-stranded nucleic acids (e.g., unhybridized nucleic acids). The latter category of methods includes intercalation of dyes, such as, for example, ethidium bromide, into double-stranded nucleic acids, differential absorbance properties of double and single stranded nucleic acids, binding of proteins that preferentially bind double-stranded nucleic acids, and the like.

### EXEMPLIFICATION

### Example 1

### Primer/Probe Generation and Evaluation

Primer/Probe Generation

The methods of the present invention determine candidate primer and probe sequences based upon the target HIV-1 sequences. The target HIV-1 sequences are genomic sequences. According to the methods of this invention, a consensus is not generated from the alignment of these genomic HIV-1 sequences. Each of the target HIV-1 genomic sequences is analyzed independently. The methods of the invention utilize an alignment of the target HIV-1 sequences to generate candidate primer and probe sequences. The methods of the present invention have the capability to determine an oligonucleotide set containing a combination of one or more probes with one or more forward and reverse primer pairs for each target sequence. The methods of the invention employ novel technology for the analysis and assessment of each primer/probe set to determine the best in silico sets. Given a number of target sequences, an in silico evaluation determines a minimal set of oligonucleotides (primers and probes) required to perfectly match every target sequence. The ideal candidate primer/probe set is one that can perform RT-PCR or PCR and the sequences are perfectly complimentary to all the known target sequences that were used to generate the alignment. The hybridization conditions for TaqMan® as an example are: 10-50 mM Tris-HCl pH 8.3, 50 mM KCl, 0.1-0.2% Triton® X-100 or 0.1% Tween®, 1-5 mM MgCl₂. The hybridization is performed at 58-70° C. for the primers and probes.

The methods of this invention include novel methods for preventing cross-reactivity between non-specific organisms and cross-reactivity between the primers and probes in the reaction.

Primer/Probe Evaluation

The candidate primers and probes are evaluated using any of a number of methods of the invention, such as, for example, secondary structure analysis.

A. Secondary Structure

The methods of the present invention include analysis of nucleic acid secondary structure. This includes the structures of the primers and/or probes, as well as their intended target variant sequences. The methods and software of the invention predict the optimal temperatures for the annealing but assumes that the target (e.g., RNA or DNA) does not have any significant secondary structure. For example, if the starting material is RNA, the first stage is the creation of a complimentary strand of DNA (cDNA) using a specific primer. This is usually performed at temperatures where the RNA template can have significant secondary structure thereby preventing the annealing of the primer. Similarly, after denaturation of a double stranded DNA target (for example, an amplicon after PCR), the binding of the probe is dependent on there being no major secondary structure in the amplicon.

The methods of the invention can either use this information as a criteria for selecting primers and probes or evaluate any secondary structure of a selected sequence, for example, by cutting and pasting candidate primer or probe sequences into a commercial internet link that uses software dedicated to analyzing secondary structure, such as, for example, MFOLD (Zuker et al. (1999) Algorithms and Thermodynamics for RNA Secondary Structure Prediction: A Practical Guide in RNA Biochemistry and Biotechnology, J. Barciszewski and B. F. C. Clark, eds., NATO ASI Series, Kluwer Academic Publishers).

B. Evaluating the Primer and Probe Sequences

The methods of the invention may also analyze any nucleic acid sequence to determine its suitability in a nucleic acid amplification-based assay.

### Example 2

### Multiplexing

The methods of the present invention enable determination of an oligonucleotide solution set comprising a combination of multiple probes (e.g., probes specific for groups M, N, and O) with at least one forward and reverse primer pair for each target sequence. For example, an oligonucleotide solution set may include one forward primer, one reverse primer and three probes (one Group M, one Group N, one Group O) specific for a target HIV-1 sequence. This oligonucleotide set is in comparison to an oligonucleotide set that only contains one forward primer, one reverse primer and one probe specific for a target sequence.

The methods of the present invention enable determination of a minimal set of oligonucleotides (primers and probes) required to perfectly match every target HIV-1 sequence. For example, if there are six target HIV-1 sequences, the methods of this invention may determine a set of five oligonucleotides (comprising, for example, two forward primers (FP#1 and FP#2), one reverse primer (RP#1) and two probes (P#1 and P#2) that will perfectly match each of the six target HIV-1 sequences in one combination or another. For instance, FP#1, RP#1 and P#1 may perfectly match target sequence one, while FP#2, RP#1 and P#1 may perfectly match target sequence two.

### Example 3

### Sequences Identified for Detecting HIV-1

A set of primers and probes useful for detecting, quantitating and grouping HIV-1 and the three groups (M, N, and O) was generated. The sets of primers and probes were then evaluated according to the methods described herein to identify the optimized primers and probes listed therein. It should be noted that the primers, as they are sequences that anneal to a plurality of all identified or unidentified HIV-1 variants, can also be used as probes either in the presence or absence of amplification of an HIV-1 sample. Each set of oligonucleotides includes at least one forward primer, at least one reverse primer, and three probes, each of which is specific for a particular group of HIV-1: M, N, or O. Particularly useful sets of forward and reverse primers and probes are described in Table 1, which employ the forward primer of SEQ ID NO:1, the reverse primers of SEQ ID NOS:2, 19, 20, 21 or 22, one probe denoted by SEQ ID NOS:4 or 6 (Group N), one probe denoted by SEQ ID NOS:5, 7 or 23 (Group O), and one probe selected from the group consisting of: SEQ ID NOS:8-14 (Group M). Based on the sequence information below, one of skill in the art would know how to create additional sets of oligonucleotides, including at least one forward primer, at least one reverse primer and one or more probes.

The sequences of the primers and probes of the present invention are:

| | |
|---|---|
| SEQ ID NO: 1: | TCAATAAAGCTTGCCTTGAG |
| | (Forward primer) |
| SEQ ID NO: 2: | TGTTCGGGCGCCACT |
| | (Reverse primer) |
| SEQ ID NO: 3: | CAATAAAGCTTGCCTTGAG |
| | (Forward primer) |
| SEQ ID NO: 4: | CCATCCATTCGGTAACTCTGGTA |
| | (Probe-Group N) |
| SEQ ID NO: 5: | TGCTCATCTGTTCAACCCTGG |
| | (Probe-Group O) |
| SEQ ID NO: 6: | CCATCCATTCGGTAACTCTGGT |
| | (Probe-Group N) |
| SEQ ID NO: 7: | TGTGCTCATCTGTTCAACCCTGT |
| | (Probe-Group O) |
| SEQ ID NO: 8: | TCTGGTAACTAGAGATCCCTCAGAC |
| | (Probe-Group M) |
| SEQ ID NO: 9: | CTGGTAACTAGAGATCCCTCAGACC |
| | (Probe-Group M) |
| SEQ ID NO: 10: | CTGGTAACTAGAGATCCCTCAGAC |
| | (Probe-Group M) |
| SEQ ID NO: 11: | TGGTAACTAGAGATCCCTCAGACC |
| | (Probe-Group M) |
| SEQ ID NO: 12: | CTCTGGTAACTAGAGATCCCTCAGACC |
| | (Probe- Group M-extended) |
| SEQ ID NO: 13: | ACTCTGGTAACTAGAGATCCCTCAGACC |
| | (Probe-Group M-extended) |
| SEQ ID NO: 14: | GACTCTGGTAACTAGAGATCCCTCAGACC |
| | (Probe-Group M-extended) |
| SEQ ID NO: 15: | CTCAATAAAGCTTGCCTTGAG |
| | (Forward primer-extended) |
| SEQ ID NO: 16: | TCAATAAAGCTTGCCTTGAGT |
| | (Forward primer-extended) |
| SEQ ID NO: 17: | CTCAATAAAGCTTGCCTTGAGT |
| | (Forward primer-extended) |
| SEQ ID NO: 18: | CCTCAATAAAGCTTGCCTTGAGTG |
| | (Forward primer-extended) |
| SEQ ID NO: 19: | TGTTCGGGCGCCACTG |
| | (Reverse primer-extended) |
| SEQ ID NO: 20: | CTGTTCGGGCGCCACT |
| | (Reverse primer-extended) |
| SEQ ID NO: 21: | CTGTTCGGGCGCCACTG |
| | (Reverse primer-extended) |
| SEQ ID NO: 22: | CCTGTTCGGGCGCCACTGC |
| | (Reverse primer-extended) |
| SEQ ID NO:23: | TGTGCTCATCTGTTCAACCCTG |
| | (Probe-Group O) |

TABLE 1

Multiplex Primers and Probes for HIV-1
Detection, Quantification and Grouping

| | | | |
|---|---|---|---|
| Set | Forward Primer | Probes (Groups M, N and O) | Reverse Primer |

| | | | | |
|---|---|---|---|---|
| 1 | SEQ ID NO: 1 | SEQ ID NO: 5 | TGCTCATCTGTTCAACCCTGG-O | SEQ ID NO: 2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 8 | TCTGGTAACTAGAGATCCCTCAGAC-M | Reverse primer |
| 2. | SEQ ID NO: 1 | SEQ ID NO: 5 | TGCTCATCTGTTCAACCCTGG-O | SEQ ID NO: 2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 9 | CTGGTAACTAGAGATCCCTCAGACC-M | Reverse primer |
| 3. | SEQ ID NO: 1 | SEQ ID NO: 5 | TGCTCATCTGTTCAACCCTGG-O | SEQ ID NO: 2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 10 | CTGGTAACTAGAGATCCCTCAGAC-M | Reverse primer |
| 4. | SEQ ID NO: 1 | SEQ ID NO: 5 | TGCTCATCTGTTCAACCCTGG-O | SEQ ID NO: 2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 11 | TGGTAACTAGAGATCCCTCAGACC-M | Reverse primer |
| 5. | SEQ ID NO: 1 | SEQ ID NO: 5 | TGCTCATCTGTTCAACCCTGG-O | SEQ ID NO:2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 12 | CTCTGGTAACTAGAGATCCCTCAGACC-M | Reverse primer |
| 6. | SEQ ID NO: 1 | SEQ ID NO: 5 | TGCTCATCTGTTCAACCCTGG-O | SEQ ID NO: 2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 13 | ACTCTGGTAACTAGAGATCCCTCAGACC-M | Reverse primer |
| 7. | SEQ ID NO: 1 | SEQ ID NO: 5 | TGCTCATCTGTTCAACCCTGG-O | SEQ ID NO: 2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 14 | GACTCTGGTAACTAGAGATCCCTCAGACC-M | Reverse primer |
| 8. | SEQ ID NO: 1 | SEQ ID NO: 5 | TGCTCATCTGTTCAACCCTGG-O | SEQ ID NO: 2 |
| | TCAATAAAGCTTGCCTTGAG | SEQ ID NO: 6 | CCATCCATTCGGTAACTCTGGT-N | TGTTCGGGCGCCACT |
| | Forward primer | SEQ ID NO: 8 | TCTGGTAACTAGAGATCCCTCAGAC-M | Reverse primer |
| 9. | SEQ ID NO: 1 | SEQ ID NO: 5 | TGCTCATCTGTTCAACCCTGG-O | SEQ ID NO: 2 |
| | TCAATAAAGCTTGCCTTGAG | SEQ ID NO: 6 | CCATCCATTCGGTAACTCTGGT-N | TGTTCGGGCGCCACT |
| | Forward primer | SEQ ID NO: 9 | CTGGTAACTAGAGATCCCTCAGACC-M | Reverse primer |
| 10. | SEQ ID NO: 1 | SEQ ID NO: 5 | TGCTCATCTGTTCAACCCTGG-O | SEQ ID NO: 2 |
| | TCAATAAAGCTTGCCTTGAG | SEQ ID NO: 6 | CCATCCATTCGGTAACTCTGGT-N | TGTTCGGGCGCCACT |
| | Forward primer | SEQ ID NO: 10 | CTGGTAACTAGAGATCCCTCAGAC-M | Reverse primer |
| 11. | SEQ ID NO: 1 | SEQ ID NO: 5 | TGCTCATCTGTTCAACCCTGG-O | SEQ ID NO: 2 |
| | TCAATAAAGCTTGCCTTGAG | SEQ ID NO: 6 | CCATCCATTCGGTAACTCTGGT-N | TGTTCGGGCGCCACT |
| | Forward primer | SEQ ID NO: 11 | TGGTAACTAGAGATCCCTCAGACC-M | Reverse primer |
| 12. | SEQ ID NO: 1 | SEQ ID NO: 5 | TGCTCATGTGTTCAACCCTGG-O | SEQ ID NO: 2 |
| | TCAATAAAGCTTGCCTTGAG | SEQ ID NO: 6 | CCATCCATTCGGTAACTCTGGT-N | TGTTCGGGCGCCACT |
| | Forward primer | SEQ ID NO: 12 | CTCTGGTAACTAGAGATCCCTCAGACC-M | Reverse primer |
| 13. | SEQ ID NO: 1 | SEQ ID NO: 5 | TGCTCATCTGTTCAACCCTGG-O | SEQ ID NO: 2 |
| | TCAATAAAGCTTGCCTTGAG | SEQ ID NO: 6 | CCATCCATTCGGTAACTCTGGT-N | TGTTCGGGCGCCACT |
| | Forward primer | SEQ ID NO: 13 | ACTCTGGTAACTAGAGATCCCTCAGACC-M | Reverse primer |
| 14. | SEQ ID NO: 1 | SEQ ID NO: 5 | TGCTCATCTGTTCAACCCTGG-O | SEQ ID NO: 2 |
| | TCAATAAAGCTTGCCTTGAG | SEQ ID NO: 6 | CCATCCATTCGGTAACTCTGGT-N | TGTTCGGGCGCCACT |
| | Forward primer | SEQ ID NO: 14 | GACTCTGGTAACTAGAGATCCCTCAGACC-M | Reverse primer |
| 15. | SEQ ID NO: 1 | SEQ ID NO: 7 | TGTGCTCATCTGTTCAACCCTGT-O | SEQ ID NO: 2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 8 | TCTGGTAACTAGAGATCCCTCAGAC-M | Reverse primer |
| 16. | SEQ ID NO: 1 | SEQ ID NO: 7 | TGTGCTCATCTGTTCAACCCTGT-O | SEQ ID NO: 2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 9 | CTGGTAACTAGAGATCCCTCAGACC-M | Reverse primer |
| 17. | SEQ ID NO: 1 | SEQ ID NO: 7 | TGTGCTCATCTGTTCAACCCTGT-O | SEQ ID NO: 2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 10 | CTGGTAACTAGAGATCCCTCAGAC-M | Reverse primer |
| 18. | SEQ ID NO: 1 | SEQ ID NO: 7 | TGTGCTCATCTGTTCAACCCTGT-O | SEQ ID NO: 2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 11 | TGGTAACTAGAGATCCCTCAGACC-M | Reverse primer |
| 19. | SEQ ID NO: 1 | SEQ ID NO: 7 | TGTGCTCATCTGTTCAACCCTGT-O | SEQ ID NO: 2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 12 | CTCTGGTAACTAGAGATCCCTCAGACC-M | Reverse primer |
| 20. | SEQ ID NO: 1 | SEQ ID NO: 7 | TGTGCTCATCTGTTCAACCCTGT-O | SEQ ID NO: 2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 13 | ACTCTGGTAACTAGAGATCCCTCAGACC-M | Reverse primer |
| 21. | SEQ ID NO: 1 | SEQ ID NO: 7 | TGTGCTCATCTGTTCAACCCTGT-O | SEQ ID NO: 2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 14 | GACTCTGGTAACTAGAGATCCCTCAGACC-M | Reverse primer |
| 22. | SEQ ID NO: 1 | SEQ ID NO: 7 | TGTGCTCATCTGTTCAACCCTGT-O | SEQ ID NO: 2 |
| | TCAATAAAGCTTGCCTTGAG | SEQ ID NO: 6 | CCATCCATTCGGTAACTCTGGT-N | TGTTCGGGCGCCACT |
| | Forward primer | SEQ ID NO: 8 | TCTGGTAACTAGAGATCCCTCAGAC-M | Reverse primer |
| 23. | SEQ ID NO: 1 | SEQ ID NO: 7 | TGTGCTCATCTGTTCAACCCTGT-O | SEQ ID NO: 2 |
| | TCAATAAAGCTTGCCTTGAG | SEQ ID NO: 6 | CCATGCATTCGGTAACTCTGGT-N | TGTTCGGGCGCCACT |
| | Forward primer | SEQ ID NO: 9 | CTGGTAACTAGAGATCCCTCAGACC-M | Reverse primer |
| 24. | SEQ ID NO: 1 | SEQ ID NO: 7 | TGTGCTCATCTGTTCAACCCTGT-O | SEQ ID NO: 2 |
| | TCAATAAAGCTTGCCTTGAG | SEQ ID NO: 6 | CCATCCATTCGGTAACTCTGGT-N | TGTTCGGGCGCCACT |
| | Forward primer | SEQ ID NO: 10 | CTGGTAACTAGAGATCCCTCAGAC-M | Reverse primer |
| 25. | SEQ ID NO: 1 | SEQ ID NO: 7 | TGTGCTCATCTGTTCAACCCTGT-O | SEQ ID NO: 2 |
| | TCAATAAAGCTTGCCTTGAG | SEQ ID NO: 6 | CCATCCATTCGGTAACTCTGGT-N | TGTTCGGGCGCCACT |
| | Forward primer | SEQ ID NO: 11 | TGGTAACTAGAGATCCCTCAGACC-M | Reverse primer |
| 26. | SEQ ID NO: 1 | SEQ ID NO: 7 | TGTGCTCATCTGTTCAACCCTGT-O | SEQ ID NO: 2 |
| | TCAATAAAGCTTGCCTTGAG | SEQ ID NO: 6 | CCATCCATTCGGTAAGTCTGGT-N | TGTTCGGGCGCCACT |
| | Forward primer | SEQ ID NO: 12 | CTCTGGTAACTAGAGATCCCTCAGACC-M | Reverse primer |
| 27. | SEQ ID NO: 1 | SEQ ID NO: 7 | TGTGCTCATCTGTTCAACCCTGT-O | SEQ ID NO: 2 |
| | TCAATAAAGCTTGCCTTGAG | SEQ ID NO: 6 | CCATCCATTCGGTAACTCTGGT-N | TGTTCGGGCGCCACT |
| | Forward primer | SEQ ID NO: 13 | ACTCTGGTAACTAGAGATCCCTCAGACC-M | Reverse primer |
| 28. | SEQ ID NO: 1 | SEQ ID NO:7 | TGTGCTCATCTGTTCAACCCTGT-O | SEQ ID NO: 2 |
| | TCAATAAAGGTTGCCTTGAG | SEQ ID NO: 6 | CCATCCATTCGGTAACTCTGGT-N | TGTTCGGGCGCCACT |
| | Forward primer | SEQ ID NO: 14 | GACTCTGGTAACTAGAGATCCCTCAGACC-M | Reverse primer |
| 29. | SEQ ID NO: 1 | SEQ ID NO: 23 | TGTGCTCATCTGTTCAAGCCTG-O | SEQ ID NO: 2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 8 | TCTGGTAACTAGAGATCCCTCAGAC-M | Reverse primer |
| 30. | SEQ ID NO: 1 | SEQ ID NO:23 | TGTGCTCATCTGTTCAACCCTG-O | SEQ ID NO: 2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 9 | CTGGTAACTAGAGATCCCTCAGACC-M | Reverse primer |
| 31. | SEQ ID NO: 1 | SEQ ID NO: 23 | TGTGCTCATCTG1TCAACCCTG-O | SEQ ID NO: 2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 10 | CTGGTAACTAGAGATCCCTCAGAC-M | Reverse primer |
| 32. | SEQ ID NO: 1 | SEQ ID NO: 23 | TGTGCTCATCTGTTCAACCCTG-O | SEQ ID NO: 2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 11 | TGGTAACTAGAGATCCCTCAGACC-M | Reverse primer |
| 33. | SEQ ID NO: 1 | SEQ ID NO: 23 | TGTGCTCATCTGTTCAACCCTG-O | SEQ ID NO: 2 |
| | TCAATAAAGGTGCCTTGAG | SEQ ID NO: 4 | CCATCCATTCGGTAACTCTGGTA-N | TGTTCGGGCGCCACT |
| | Forward primer | SEQ ID NO: 12 | CTCTGGTAACTAGAGATCCCTCAGACC-M | Reverse primer |
| 34. | SEQ ID NO: 1 | SEQ ID NO: 23 | TGTGCTCATCTGTTCAACCCTG-O | SEQ ID NO: 2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 13 | ACTCTGGTAACTAGAGATCCCTCAGACC-M | Reverse primer |
| 35. | SEQ ID NO: 1 | SEQ ID NO: 23 | TGTGCTCATCTGTTCAACCCTG-O | SEQ ID NO: 2 |
| | TCAATAAAGCTTGCCTTGAG | SEQ ID NO: 4 | CCATCCATTCGGTAACTCTGGTA-N | TGTTCGGGCGCCACT |
| | Forward primer | SEQ ID NO: 14 | GACTCTGGTAACTAGAGATCCCTCAGACC-M | Reverse primer |
| 36. | SEQ ID NO: 1 | SEQ ID NO: 23 | TGTGCTCATCTGTTCAACCCTG-O | SEQ ID NO: 2 |
| | TCAATAAAGCTTGCCTTGAG | SEQ ID NO: 6 | CCATCCATTCGGTAACTCTGGT-N | TGTTCGGGCGCCACT |
| | Forward primer | SEQ ID NO: 8 | TCTGGTAACTAGAGATCCCTCAGAC-M | Reverse primer |
| 37. | SEQ ID NO: 1 | SEQ ID NO: 23 | TGTGCTCATCTGTTCAACCCTG-O | SEQ ID NO: 2 |
| | TCAATAAAGCTTGCCTTGAG | SEQ ID NO: 6 | CCATCCATTCGGTAACTCTGGT-N | TGTTCGGGCGCCACT |
| | Forward primer | SEQ ID NO: 9 | CTGGTAACTAGAGATCCCTCAGACC-M | Reverse primer |
| 38. | SEQ ID NO: 1 | SEQ ID NO: 23 | TGTGCTCATCTGTTCAACCCTG-O | SEQ ID NO: 2 |
| | TCAATAAAGCTTGCCTTGAG | SEQ ID NO: 6 | CCATCCATTCGGTAACTCTGGT-N | TGTTCGGGCGCCACT |
| | Forward primer | SEQ ID NO: 10 | CTGGTAACTAGAGATCCCTCAGAC-M | Reverse primer |
| 39. | SEQ ID NO: 1 | SEQ ID NO: 23 | TGTGGTCATCTGTTCAACCCTG-O | SEQ ID NO: 2 |
| | TCAATAAAGCTTGCCTTGAG | SEQ ID NO: 6 | CCATCCATTCGGTAACTCTGGT-N | TGTTCGGGCGCCACT |
| | Forward primer | SEQ ID NO: 11 | TGGTAACTAGAGATCCCTCAGACC-M | Reverse primer |
| 40. | SEQ ID NO: 1 | SEQ ID NO: 23 | TGTGCTCATCTGTTCAACCCTG-O | SEQ ID NO: 2 |
| | TCAATAAAGCTTGCCTTGAG | SEQ ID NO: 6 | CCATCCATTCGGTAACTCTGGT-N | TGTTCGGGCGCCACT |
| | Forward primer | SEQ ID NO: 12 | CTCTGGTAACTAGAGATCCCTCAGACC-M | Reverse primer |
| 41. | SEQ ID NO: 1 | SEQ ID NO: 23 | TGTGCTCATCTGTTCAACCCTG-O | SEQ ID NO: 2 |
| | TCAATAAAGCTTGCCTTGAG | SEQ ID NO: 6 | CCATCCATTCGGTAACTCTGGT-N | TGTTCGGGCGCCACT |
| | Forward primer | SEQ ID NO: 13 | ACTCTGGTAACTAGAGATCCCTCAGACC-M | Reverse primer |
| 42. | SEQ ID NO: 1 | SEQ ID NO: 23 | TGTGCTCATCTGTTCAACCCTG-O | SEQ ID NO: 2 |
| | TCAATAAAGCTTGCCTTGAG | SEQ ID NO: 6 | CCATCCA1TCGGTAACTCTGGT-N | TGTTCGGGCGCCACT |
| | Forward primer | SEQ ID NO: 14 | GACTCTGGTAACTAGAGATCCCTCAGACC-M | Reverse primer |
| 43. | SEQ ID NO: 1 | SEQ ID NO: 5 | TGCTCATCTGTTCAACCCTGG-O | SEQ ID NO: 19 |
| | TCAATAAAGCTTGCCTTGAG | SEQ ID NO: 4 | CCATCCATTCGGTAACTCTGGTA-N | TGTTCGGGCGCCACTG |
| | Forward primer | SEQ ID NO: 8 | TCTGGTAACTAGAGATCCCTCAGAC-M | Reverse primer-extended |
| 44. | SEQ ID NO: 1 | SEQ ID NO: 5 | TGCTCATCTGTTCAACCCTGG-O | SEQ ID NO: 19 |
| | TCAATAAAGCTTGCCTTGAG | SEQ ID NO: 4 | CCATCCATTCGGTAACTCTGGTA-N | TGTTCGGGCGCCACTG |
| | Forward primer | SEQ ID NO: 9 | CTGGTAAGTAGAGATCCCTCAGACC-M | Reverse primer-extended |
| 45. | SEQ ID NO: 1 | SEQ ID NO: 5 | TGCTCATCTGTTCAACCCTGG-O | SEQ ID NO: 19 |
| | TCAATAAAGCTTGCCTTGAG | SEQ ID NO: 4 | CCATCCATTCGGTAACTCTGGTA-N | TGTTCGGGCGCCACTG |
| | Forward primer | SEQ ID NO: 10 | CTGGTAACTAGAGATCCCTCAGAC-M | Reverse primer-extended |
| 46. | SEQ ID NO: 1 | SEQ ID NO: 5 | TGCTCATCTGTTCAACCCTGG-O | SEQ ID NO: 19 |
| | TCAATAAAGCTTGCCTTGAG | SEQ ID NO: 4 | CCATCCATTCGGTAACTCTGGTA-N | TGTTCGGGCGCCACTG |
| | Forward primer | SEQ ID NO: 11 | TGGTAACTAGAGATCCCTCAGACC-M | Reverse primer-extended |
| 47. | SEQ ID NO: 1 | SEQ ID NO: 5 | TGCTCATCTGTTCAACCCTGG-O | SEQ ID NO: 19 |
| | TCAATAAAGCTTGCCTTGAG | SEQ ID NO: 4 | CCATCCATTCGGTAACTCTGGTA-N | TGTTGGGGCGCCACTG |
| | Forward primer | SEQ ID NO: 12 | CTCTGGTAACTAGAGATCCCTCAGACC-M | Reverse primer-extended |
| 48. | SEQ ID NO: 1 | SEQ ID NO: 5 | TGCTCATCTGTTCAACCCTGG-O | SEQ ID NO: 19 |
| | TCAATAAAGCTTGGCTTGAG | SEQ ID NO: 4 | CCATCCATTCGGTAACTCTGGTA-N | TGTTCGGGCGCCACTG |
| | Forward primer | SEQ ID NO: 13 | ACTCTGGTAACTAGAGATCCCTCAGACC-M | Reverse primer-extended |
| 49. | SEQ ID NO: 1 | SEQ ID NO: 5 | TGCTCATCTGTTCAACCCTGG-O | SEQ ID NO: 19 |
| | TCAATAAAGCTTGCCTTGAG | SEQ ID NO: 4 | CCATCCATTCGGTAACTCTGGTA-N | TGTTCGGGCGCCACTG |
| | Forward primer | SEQ ID NO: 14 | GACTCTGGTAACTAGAGATCCCTCAGACC-M | Reverse primer-extended |
| 50. | SEQ ID NO: 1 | SEQ ID NO: 5 | TGCTCATCTGTTCAACCCTGG-O | SEQ ID NO: 19 |
| | TCAATAAAGCTTGCCTTGAG | SEQ ID NO: 6 | CCATCCATTCGGTAACTCTGGT-N | TGTTCGGGCGCCACTG |
| | Forward primer | SEQ ID NO: 8 | TCTGGTAACTAGAGATCCCTCAGAC-M | Reverse primer-extended |
| 51. | SEQ ID NO: 1 | SEQ ID NO: 5 | TGCTCATCTGTTCAACCCTGG-O | SEQ ID NO: 19 |
| | TCAATAAAGCTTGCCTTGAG | SEQ ID NO: 6 | CCATCCATTCGGTAACTCTGGT-N | TGTTCGGGCGCCACTG |
| | Forward primer | SEQ ID NO: 9 | CTGGTAACTAGAGATCCCTCAGACC-M | Reverse primer-extended |
| 52. | SEQ ID NO: 1 | SEQ ID NO: 5 | TGCTCATCTGTTCAACCCTGG-O | SEQ ID NO: 19 |
| | TCAATAAAGCTTGCCTTGAG | SEQ ID NO: 6 | CCATCCATTCGGTAACTCTGGT-N | TGTTCGGGCGCCACTG |
| | Forward primer | SEQ ID NO: 10 | CTGGTAACTAGAGATCCCTCAGAC-M | Reverse primer-extended |
| 53. | SEQ ID NO: 1 | SEQ ID NO: 5 | TGCTCATCTGTTCAACCCTGG-O | SEQ ID NO: 19 |
| | TCAATAAAGCTTGCCTTGAG | SEQ ID NO: 6 | CCATCCATTCGGTAACTCTGGT-N | TGTTCGGGCGCCACTG |
| | Forward primer | SEQ ID NO: 11 | TGGTAACTAGAGATCCCTCAGACC M | Reverse primer-extended |
| 54. | SEQ ID NO: 1 | SEQ ID NO: 5 | TGCTGATCTGTTCAACCCTGG-O | SEQ ID NO: 19 |
| | TCAATAAAGGTTGCCTTGAG | SEQ ID NO: 6 | CCATCCATTCGGTAAGTCTGGT-N | TGTTCGGGCGCCACTG |
| | Forward primer | SEQ ID NO: 12 | CTCTGGTAACTAGAGATCCCTCAGACC-M | Reverse primer-extended |
| 55. | SEQ ID NO: 1 | SEQ ID NO: 5 | TGCTCATCTGTTCAACCCTGG-O | SEQ ID NO: 19 |
| | TCAATAAAGCTTGCCTTGAG | SEQ ID NO: 6 | CCATCCATTCGGTAACTCTGGT-N | TGTTCGGGCGCCACTG |
| | Forward primer | SEQ ID NO: 13 | ACTCTGGTAACTAGAGATCCCTCAGACC-M | Reverse primer-extended |
| 56. | SEQ ID NO:1 | SEQ ID NO: 5 | TGCTCATCTGTTCAACCCTGG-O | SEQ ID NO: 19 |
| | TCAATAAAGGTTGCCTTGAG | SEQ ID NO: 6 | CCATCCATTCGGTAACTCTGGT-N | TGTTCGGGCGCCACTG |
| | Forward primer | SEQ ID NO: 14 | GACTCTGGTAACTAGAGATCCCTCAGACC-M | Reverse primer-extended |
| 57. | SEQ ID NO: 1 | SEQ ID NO: 7 | TGTGCTCATCTGTTCAACCCTGT-O | SEQ ID NO: 19 |
| | TCAATAAAGCTTGCCTTGAG | SEQ ID NO: 4 | CCATCCATTCGGTAACTCTGGTA-N | TGTTCGGGCGCCACTG |
| | Forward primer | SEQ ID NO: 8 | TCTGGTAACTAGAGATCCCTCAGAC-M | Reverse primer-extended |
| 58. | SEQ ID NO: 1 | SEQ ID NO: 7 | TGTGCTCATCTGTTCAACCCTGT-O | SEQ ID NO: 19 |
| | TCAATAAAGCTTGCCTTGAG | SEQ ID NO: 4 | CCATCCATTCGGTAACTCTGGTA-N | TGTTCGGGCGCCACTG |
| | Forward primer | SEQ ID NO: 9 | CTGGTAACTAGAGATCCCTCAGACC-M | Reverse primer-extended |
| 59. | SEQ ID NO: 1 | SEQ ID NO: 7 | TGTGCTCATCTGTTCAACCCTGT-O | SEQ ID NO: 19 |
| | TCAATAAAGCTTGCCT5GAG | SEQ ID NO: 4 | CCATCCATTCGGTAACTCTGGTA-N | TGTTCGGGCGCCACTG |
| | Forward primer | SEQ ID NO: 10 | CTGGTAACTAGAGATCCCTCAGAC-M | Reverse primer-extended |
| 60. | SEQ ID NO: 1 | SEQ ID NO: 7 | TGTGCTCATCTGTTCAACCCTGT-O | SEQ ID NO: 19 |
| | TCAATAAAGCTTGCCTTGAG | SEQ ID NO: 4 | CCATCCATTCGGTAACTCTGGTA-N | TGTTCGGGCGCCACTG |
| | Forward primer | SEQ ID NO: 11 | TGGTAACTAGAGATCCCTCAGACC-M | Reverse primer-extended |
| 61. | SEQ ID NO: 1 | SEQ ID NO: 7 | TGTGCTCATCTGTTCAACCCTGT-O | SEQ ID NO: 19 |
| | TCAATAAAGCTTGCCTTGAG | SEQ ID NO: 4 | CCATCCATTCGGTAACTCTGGTA-N | TGTTCGGGCGCCACTG |
| | Forward primer | SEQ ID NO: 12 | CTCTGGTAACTAGAGATCCCTCAGACC-M | Reverse primer-extended |
| 62. | SEQ ID NO: 1 | SEQ ID NO: 7 | TGTGCTCATCTGTTCAACCCTGT-O | SEQ ID NO: 19 |
| | TCAATAAAGGTTGCCTTGAG | SEQ ID NO: 4 | CCATCCATTCGGTAACTCTGGTA-N | TGTTCGGGCGCCACTG |
| | Forward primer | SEQ ID NO: 13 | ACTCTGGTAACTAGAGATCCCTCAGACC-M | Reverse primer-extended |
| 63. | SEQ ID NO: 1 | SEQ ID NO: 7 | TGTGCTCATCTGTTCAACCCTGT-O | SEQ ID NO: 19 |
| | TCAATAAAGCTTGCCTTGAG | SEQ ID NO: 4 | CCATCCATTCGGTAACTCTGGTA-N | TGTTCGGGCGCCACTG |
| | Forward primer | SEQ ID NO: 14 | GACTCTGGTAACTAGAGATCCCTCAGACC-M | Reverse primer-extended |
| 64. | SEQ ID NO: 1 | SEQ ID NO: 7 | TGTGCTCATCTGTTCAACCCTGT-O | SEQ ID NO: 19 |
| | TCAATAAAGCTTGCCTTGAG | SEQ ID NO: 6 | CCATCCATTCGGTAACTCTGGT-N | TTTCGGGCGCCACTG |
| | Forward primer | SEQ ID NO: 8 | TCTGGTAACTAGAGATCCCTCAGAC-M | Reverse primer-extended |
| 65. | SEQ ID NO: 1 | SEQ ID NO: 7 | TGTGCTCATCTGTTCAACCCTGT-O | SEQ ID NO: 19 |
| | TCAATAAAGCTTGCCTTGAG | SEQ ID NO: 6 | CCATCCATTCGGTAACTCTGGT-N | TGTTCGGGCGCCACTG |
| | Forward primer | SEQ ID NO: 9 | CTGGTAACTAGAGATCCCTCAGACC-M | Reverse primer-extended |
| 66. | SEQ ID NO: 1 | SEQ ID NO: 7 | TGTGCTGATCTGTTCAACCCTGT-O | SEQ ID NO: 19 |
| | TCAATAAAGCTTGCCTTGAG | SEQ ID NO: 6 | CCATCCATTCGGTAACTCTGGT-N | TGTTCGGGCGCCACTG |
| | Forward primer | SEQ ID NO: 10 | CTGGTAACTAGAGATCCCTCAGAC-M | Reverse primer-extended |
| 67. | SEQ ID NO: 1 | SEQ ID NO: 7 | TGTGCTCATCTGTTCAACCCTGT-O | SEQ ID NO: 19 |
| | TCAATAAAGCTTGCCTTGAG | SEQ ID NO: 6 | CCATCCATTCGGTAACTCTGGT-N | TGTTCGGGCGCCACTG |
| | Forward primer | SEQ ID NO: 11 | TGGTAACTAGAGATCCCTCAGACC-M | Reverse primer-extended |
| 68. | SEQ ID NO: 1 | SEQ ID NO: 7 | TGTGCTCATCTGTTCAACCCTGT-O | SEQ ID NO: 19 |
| | TCAATAAAGCTTGCCTTGAG | SEQ ID NO: 6 | CCATCCATTCGGTAACTCTGGT-N | TGTTCGGGCGCCACTG |
| | Forward primer | SEQ ID NO: 12 | CTCTGGTAACTAGAGATCCCTCAGACC-M | Reverse primer-extended |
| 69. | SEQ ID NO: 1 | SEQ ID NO: 7 | TGTGCTCATCTGTTCAACCCTGT-O | SEQ ID NO: 19 |
| | TCAATAAAGCTTGCCTTGAG | SEQ ID NO: 6 | CCATCCATTCGGTAACTCTGGT-N | TGTTCGGGCGCCACTG |
| | Forward primer | SEQ ID NO: 13 | ACTCTGGTAACTAGAGATCCCTCAGACC-M | Reverse primer-extended |
| 70. | SEQ ID NO: 1 | SEQ ID NO: 7 | TGTGCTCATCTGTTCAACCCTGT-O | SEQ ID NO: 19 |
| | TCAATAAAGCTTGCCTTGAG | SEQ ID NO: 6 | CCATCCATTCCTGTAACTCTGGT-N | TGTTCGGGCGCCACTG |
| | Forward primer | SEQ ID NO: 14 | GACTCTGGTAACTAGAGATCCCTCAGACC-M | Reverse primer-extended |
| 71. | SEQ ID NO: 1 | SEQ ID NO: 23 | TGTGCTCATCTGTTCAACCCTG-O | SEQ ID NO: 19 |
| | TCAATAAAGCTTGCCTTGAG | SEQ ID NO: 4 | CCATCCATTCGGTAACTCTGGTA-N | TGTTCGGGCGCCACTG |
| | Forward primer | SEQ ID NO: 8 | TCTGGTAACTAGAGATCCCTCAGAC- M | Reverse primer-extended |
| 72. | SEQ ID NO: 1 | SEQ ID NO: 23 | TGTGCTCATCTGTTCAAGCCTG-O | SEQ ID NO: 19 |
| | TCAATAAAGCTTGCCTTGAG | SEQ ID NO: 4 | CCATCCAITCGGTAACTCTGGTA-N | TGTTCGGGGGCCACTG |
| | Forward primer | SEQ ID NO: 9 | CTGGTAACTAGAGATCCCTCAGACC-M | Reverse primer-extended |
| 73. | SEQ ID NO: 1 | SEQ ID NO: 23 | TGTGCTCATCTGTTCAACCCTG-O | SEQ ID NO: 19 |
| | TCAATAAAGCTTGCCTTGAG | SEQ ID NO: 4 | CCATCCATTCGGTAACTCTGGTA-N | TGTTCGGGCGCGACTG |
| | Forward primer | SEQ ID NO: 10 | CTGGTAACTAGAGATCCCTCAGAC-M | Reverse primer-extended |
| 74. | SEQ ID NO: 1 | SEQ ID NO: 23 | TGTGCTCATCTGTTCAACCCTG-O | SEQ ID NO: 19 |
| | TCAATAAAGCTTGCCTTGAG | SEQ ID NO: 4 | CCATCCA1TCGGTAACTCTGGTA-N | TGTTCGGGCGCCACTG |
| | Forward primer | SEQ ID NO: 11 | TGGTAACTAGAGATCCCTCAGACC-M | Reverse primer-extended |
| 75. | SEQ ID NO: 1 | SEQ ID NO: 23 | TGTGCTCATCTGTTCAACCCTG-O | SEQ ID NO: 19 |
| | TCAATAAAGCTTGCCTTGAG | SEQ ID NO: 4 | CCATCCATTCGGTAACTCTGGTA-N | TGTTCGGGCGCCACTG |
| | Forward primer | SEQ ID NO: 12 | CTCTGGTAACTAGAGATCCCTCAGACC-M | Reverse primer-extended |
| 76. | SEQ ID NO: 1 | SEQ ID NO: 23 | TGTGCTCATGTGTTCAACCCTG-O | SEQ ID NO: 19 |
| | TCAATAAAGCTTGGCTTGAG | SEQ ID NO: 4 | CCATCCAATCGGTAACTCTGGTA-N | TGTTCGGGCGCCACTG |
| | Forward primer | SEQ ID NO: 13 | ACTCTGGTAACTAGAGATCCCTCAGACC-M | Reverse primer-extended |
| 77. | SEQ ID NO: 1 | SEQ ID NO: 23 | TGTGCTCATGTGTTCAACCCTG-O | SEQ ID NO: 19 |
| | TCAATAAAGCTTGCGTTGAG | SEQ ID NO: 4 | CCATCCATTCGGTAACTCTGGTA-N | TGTTCGGCTCGCCACTG |
| | Forward primer | SEQ ID NO: 14 | CIACTCTGGTAACTAGAGATCCCTCAGACC-M | Reverse primer-extended |
| 78. | SEQ ID NO: 1 | SEQ ID NO: 23 | TGTGCTCATCTGTTCAACCCTG-O | SEQ ID NO: 19 |
| | TCAATAAAGCTTGCCTTGAG | SEQ ID NO: 6 | CCATCCATTCGGTAACTCTGGT-N | TGTTCGGGCGCCACTG |
| | Forward primer | SEQ ID NO: 8 | TCTGGTAACTAGAGATCCCTCAGAC-M | Reverse primer-extended |
| 79. | SEQ ID NO: 1 | SEQ ID NO: 23 | TGTGCTCATCTGTTCAACCCTG-O | SEQ ID NO: 19 |
| | TCAATAAAGCTTGCCTTGAG | SEQ ID NO: 6 | CCATCCATTCGGTAACTCTGGT-N | TGTTCGGGCGCCACTG |
| | Forward primer | SEQ ID NO: 9 | CTGGTAACTAGAGATCCCTCAGACC-M | Reverse primer-extended |
| 80. | SEQ ID NO: 1 | SEQ ID NO: 23 | TGTGCTCATCTGTTCAACCCTG-O | SEQ ID NO: 19 |
| | TCAATAAAGGTTGCCTTGAG | SEQ ID NO: 6 | CCATCCATTCGGTAAGTCTGGT-N | TGTTCGGGCGCCACTG |
| | Forward primer | SEQ ID NO: 10 | CTGGTAACTAGAGATCCCTCAGAC-M | Reverse primer-extended |
| 81. | SEQ ID NO: 1 | SEQ ID NO: 23 | TGTGGTCATCTGTTCAACCCTG-O | SEQ ID NO: 19 |
| | TCAATAAAGCTTGCCTTGAG | SEQ ID NO: 6 | CCATCCATTCGGTAACTCTGGT-N | TGTTCGGGCGCCACTG |
| | Forward primer | SEQ ID NO: 11 | TGGTAACTAGAGATCCCTCAGACC-M | Reverse primer-extended |
| 82. | SEQ ID NO: 1 | SEQ ID NO: 23 | TGTGCTCATCTGTTCAACCCTG-O | SEQ ID NO: 19 |
| | TGAATAAAGCTTGCCTTGAG | SEQ ID NO: 6 | CCATCCATTCGGTAACTCTGGT-N | TGTTCGGGCGCCACTG |
| | Forward primer | SEQ ID NO: 12 | CTCTGGTAACTAGAGATCCCTCAGACC-M | Reverse primer-extended |
| 83. | SEQ ID NO: 1 | SEQ ID NO: 23 | TGTGCTCATCTGTTGAACCCTG-O | SEQ ID NO: 19 |
| | TCAATAAAGCTTGCCTTGAG | SEQ ID NO: 6 | CCATCCATTCGGTAACTCTGGT-N | TGTTCGGGCGCCACTG |
| | Forward primer | SEQ ID NO: 13 | ACTCTGGTAACTAGAGATCCCTCAGACC-M | Reverse primer-extended |
| 84. | SEQ ID NO: 1 | SEQ ID NO:23 | TGTGCTCATCTGTTCAACCCTG-O | SEQ ID NO: 19 |
| | TCAATAAAGCTTGCCTTGAG | SEQ ID NO: 6 | CCATGCATTCGGTAACTCTGGT-N | TGTTCGGGCGCCACTG |
| | Forward primer | SEQ ID NO: 14 | GACTCTGGTAACTAGAGATCCCTCAGACC-M | Reverse primer-extended |
| 85. | SEQ ID NO: 1 | SEQ ID NO: 5 | TGCTGATCTGTTCAACCCTGG-O | SEQ ID NO: 20 |
| | TCAATAAAGCTTGCCTTGAG | SEQ ID NO: 4 | CCATCCATTGGGTAACTCTGGTA-N | CTGTTCGGGCGCGACT |
| | Forward primer | SEQ ID NO: 8 | TCTGGTAACTAGAGATCCCTCAGAC-M | Reverse primer-extended |
| 86. | SEQ ID NO: 1 | SEQ ID NO: 5 | TGCTCATCTGTTCAACCCTGG-O | SEQ ID NO: 20 |
| . | | | | |
| | TCAATAAAGCTTGCCTTGAG | SEQ ID NO: 4 | CCATCCATTCGGTAACTCTGGTA-N | CTGTTCGGGCGCCACT |
| | Forward primer | SEQ ID NO: 9 | CTGGTAACTAGAGATCCCTCAGACC-M | Reverse primer-extended |
| 87. | SEQ ID NO: 1 | SEQ ID NO: 5 | TGCTCATCTGTTCAACCCTGG-O | SEQ ID NO: 20 |
| | TCAATAAAGCTTGCCTTGAG | SEQ ID NO: 4 | CCATCCATTCGGTAACTCTGGTA-N | CTGTTCGGGCGCGACT |
| | Forward primer | SEQ ID NO: 10 | CTGGTAACTAGAGATCCCTCAGAC-M | Reverse primer-extended |
| 88. | SEQ ID NO: 1 | SEQ ID NO: 5 | TGCTCATCTGTTCAACCCTGG-O | SEQ ID NO: 20 |
| | TCAATAAAGCTTGCCTTGAG | SEQ ID NO: 4 | CCATCCATTCGGTAACTCTGGTA-N | CTGTTCGGGCGCCACT |
| | Forward primer | SEQ ID NO: 11 | TOGTAACTAGAGATCCCTCAGACC-M | Reverse primer-extended |
| 89. | SEQ ID NO: 1 | SEQ ID NO: 5 | TGCTCATCTGTTCAACCCTGG-O | SEQ ID NO: 20 |
| | TCAATAAAGCTTGCCTTGAG | SEQ ID NO: 4 | CCATCCATTCGGTAACTCTGGTA-N | CTGTTCGGGCGCCACT |
| | Forward primer | SEQ ID NO: 12 | CTCTGGTAACTAOAGATCCCTCAGACC-M | Reverse primer-extended |
| 90. | SEQ ID NO: 1 | SEQ ID NO: 5 | TGCTCATCTGTTCAACCCTGG-O | SEQ ID NO: 20 |
| | TCAATAAAGCTTGCCTTGAG | SEQ ID NO: 4 | CCATCCATTCGGTAACTCTGGTA-N | CTGTTCGGGCGCCACT |
| | Forward primer | SEQ ID NO: 13 | ACTCTGGTAACTAGAGATCCCTCAGACC-M | Reverse primer-extended |
| 91. | SEQ ID NO: 1 | SEQ ID NO: 5 | TGCTCATCTGTTCAACCCTGG-O | SEQ ID NO: 20 |
| | TCAATAAAGGTTGCCTITGAG | SEQ ID NO: 4 | CCATCCATTCGGTAACTCTGGTA-N | CTGTTCGGGCGCCACT |
| | Forward primer | SEQ ID NO: 14 | GACTCTGGTAACTAGAGATCCGTCAGACC-M | Reverse primer-extended |
| 92. | SEQ ID NO: 1 | SEQ ID NO: 5 | TGCTCATCTG1TCAACCCTGG-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 8 | TCTGGTAACTAGAGATCCCTCAGAC-M | Reverse primer-extended |
| 93. | SEQ ID NO: 1 | SEQ ID NO: 5 | TGCTCATCTGTrCAACCCTGG-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 9 | CTGGTAACTAGAGATCCCTCAGACC-M | Reverse primer-extended |
| 94. | SEQ ID NO: 1 | SEQ ID NO: 5 | TGCTCATCTGITCAACCCTGG-O | SEQ ID NO: 20 |
| | TCAATAAAGCTTGCCTTGAG | SEQ ID NO: 6 | CCATCCATTCGGTAACTCTGGT-N | GTGTTCGGGCGCCACT |
| | Forward primer | SEQ ID NO: 10 | CTGGTAACTAGAGATCCCTCAGAC-M | Reverse primer-extended |
| 95. | SEQ ID NO: 1 | SEQ ID NO: 5 | TGCTCATCTCflTCAACCCTGG-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 11 | TGGTAACTAGAGATCCCTCAGACC-M | Reverse primer-extended |
| 96. | SEQ ID NO: 1 | SEQ ID NO: 5 | TGCTCATCTGTrCAACCCTOG-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 12 | CTCTGGTAACTAGAGATCCCTCAGACC-M | Reverse primer-extended |
| 97. | SEQ ID NO: 1 | SEQ ID NO: 5 | TGCTCATCTGTTCAACCCTGG-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 13 | ACTCTGGTAACTAGAGATCCCTCAGACC-M | Reverse primer-extended |
| 98. | SEQ ID NO: 1 | SEQ ID NO: 5 | TGCTCATCTGTTCAACCCTGG-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 14 | GACTCTGGTAACTAGAGATCCCTCAGACC-M | Reverse primer-extended |
| 99. | SEQ ID NO: 1 | SEQ ID NO: 7 | TGTGCTCATCTGTTCAACCCTGT-O | SEQ ID NO: 20 |
| | TCAATAAAGCGTGCCTTGAG | SEQ ID NO: 4 | CCATCCATTCGGTAACTCTGGTA-N | CTGTTCGGGCGCCACT |
| | Forward primer | SEQ ID NO: 8 | TCTGGTAACTAGAGATCCCTCAGAC-M | Reverse primer-extended |
| 100 | SEQ ID NO: 1 | SEQ ID NO: 7 | TGTGCTCATCTGTTCAACCCTGT-O | SEQ ID NO: 20 |
| | TCAATAAAGCTTGCCTTGAG | SEQ ID NO: 4 | CCATCCATTCGGTAACTCTGGTA-N | CTGTTCGGGCGCCACT |
| | Forward primer | SEQ ID NO: 9 | CTGGTAACTAGAGATCCCTCAGACC-M | Reverse primer-extended |
| 101 | SEQ ID NO: 1 | SEQ ID NO: 7 | TGTGCTCATCTGTTCAACCCTGT-O | SEQ ID NO: 20 |
| | TCAATAAAGCTTGCCTTGAG | SEQ ID NO: 4 | CCATCCATTCGGTAACTCTGGTA-N | CTGTTCGGGCGCCACT |
| | Forward primer | SEQ ID NO: 10 | CTGGTAACTAGAGATCCCTCAGAC-M | Reverse primer-extended |
| 102 | SEQ ID NO: 1 | SEQ ID NO: 7 | TGTGCTCATCTGTTCAACCCTGT-O | SEQ ID NO: 20 |
| | TCAATAAAGCTTGCCTTGAG | SEQ ID NO: 4 | CCATCCATTCGGTAACTCTGGTA-N | CTGTTCGGGCGCCACT |
| | Forward primer | SEQ ID NO: 11 | TGGTAACTAGAGATCCCTGAGACC-M | Reverse primer-extended |
| 103 | SEQ ID NO: 1 | SEQ ID NO: 7 | TGTGCTCATCTGTTCAACCCTGT-O | SEQ ID NO: 20 |
| | TCAATAAAGCTTGCCTTGAG | SEQ ID NO: 4 | CCATCCATTCGGTAACTCTGGTA-N | CTGTTCGGGCGCCACT |
| | Forward primer | SEQ ID NO: 12 | CTCTGGTAACTAGAGATCCCTCAGACC-M | Reverse primer-extended |
| 104 | SEQ ID NO: 1 | SEQ ID NO: 7 | TGTGCTCATCTGTTCAACCCTGT-O | SEQ ID NO: 20 |
| | TCAATAAAGCTTGCCTTGAG | SEQ ID NO: 4 | CCATCCATTCGGTAACTCTGGTA-N | CTGTTCGGGCGCCACT |
| | Forward primer | SEQ ID NO: 13 | ACTCTGGTAACTAGAGATCCCTCAGACC-M | Reverse primer-extended |
| 105 | SEQ ID NO: 1 | SEQ ID NO: 7 | TGTGCTCATCTGTTCAACCCTGT-O | SEQ ID NO: 20 |
| | TCAATAAAGCTTGCCTTGAG | SEQ ID NO: 4 | CCATCCATTCGGTAACTCTGGTA-N | CTGTTGGGGCGCCACT |
| | Forward primer | SEQ ID NO: 14 | GACTCTGGTAACTAGAGATCCCTCAGACC-M | Reverse primer-extended |
| 106 | SEQ ID NO: 1 | SEQ ID NO: 7 | TGTGCTCATCTGTTCAACCCTGT-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 8 | TCTGGTAACTAGAGATCCCTCAGAC-M | Reverse primer-extended |
| 107 | SEQ ID NO: 1 | SEQ ID NO: 7 | TGTGCTCATCTGTTCAACCCTGT-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 9 | CTGGTAACTAGAGATCCCTCAGACC-M | Reverse primer-extended |
| 108 | SEQ ID NO: 1 | SEQ ID NO: 7 | TGTGCTCATCTGTTCAACCCTGT-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 10 | CTGGTAACTAGAGATCCCTCAGAC-M | Reverse primer-extended |
| 109 | SEQ ID NO: 1 | SEQ ID NO: 7 | TGTGCTCATCTGTTCAACCCTGT-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 11 | TGGTAACTAGAGATCCCTCAGACC-M | Reverse primer-extended |
| 110 | SEQ ID NO: 1 | SEQ ID NO: 7 | TGTGCTCATCTGTTCAACCCTGT-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 12 | CTCTGGTAACTAGAGATCCCTCAGACC-M | Reverse primer-extended |
| 111 | SEQ ID NO: 1 | SEQ ID NO: 7 | TGTGCTCATCTGTTCAACCCTGT-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 13 | ACTCTGGTAACTAGAGATCCCTCAGACC-M | Reverse primer-extended |
| 112 | SEQ ID NO: 1 | SEQ ID NO: 7 | TGTGCTCATCTGTTCAACCCTGT-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 14 | GACTCTGGTAACTAGAGATCCCTCAGACC-M | Reverse primer-extended |
| 113 | SEQ ID NO: 1 | SEQ ID NO: 23 | TGTGCTCATCTGTTCAACCCTG-O | SEQ ID NO: 20 |
| | TCAATAAAGCTTGCCTTGAG | SEQ ID NO: 4 | CCATCCATTCGGTAACTCTGGTA-N | CTGTTCGGGCGCCACT |
| | Forward primer | SEQ ID NO: 8 | TCTGGTAACTAGAGATCCCTCAGAC-M | Reverse primer-extended |
| 114 | SEQ ID NO: 1 | SEQ IDNO: 23 | TGTGCTCATCTGTTCAACCCTG-O | SEQ ID NO: 20 |
| | TCAATAAAGGTTGGCTTGAG | SEQ ID NO: 4 | CCATCGATTCGGTAACTCTGGTA-N | CTGTTCGGGCGCCACT |
| | Forward primer | SEQ ID NO: 9 | CTGGTAACTAGAGATCCCTCAGACC-M | Reverse primer extended |
| 115 | SEQ ID NO: 1 | SEQ ID NO: 23 | TGTGCTCATCTGTTCAACCCTG-O | SEQ ID NO: 20 |
| | TCAATAAAGCTTGCCTTGAG | SEQ ID NO: 4 | CCATCCATTCGGTAACTCTGGTA-N | CTGTTCGGGCGCCACT |
| | Forward primer | SEQ ID NO: 10 | CTGGTAACTAGAGATCCCTCAGAC-M | Reverse primer-extended |
| 116 | SEQ ID NO: 1 | SEQ ID NO: 23 | TGTGCTCATCTGTTCAACCCTG-O | SEQ ID NO: 20 |
| | TCAATAAAGCTTGCCTTGAG | SEQ ID NO: 4 | CCATCCATTCGGTAACTCTGGTA-N | CTGTTCGGGCGGCACT |
| | Forward primer | SEQ ID NO: 11 | TGGTAACTAGAGATCCCTCAGACC-M | Reverse primer-extended |
| 117 | SEQ ID NO: 1 | SEQ ID NO: 23 | TGTGCTCATCTGTTCAACCCTG-O | SEQ ID NO: 20 |
| | TCAATAAAGCTTGCCTTGAG | SEQ ID NO: 4 | CCATCCATTCGGTAACTCTGGTA-N | CTGTTCGGGCGCCACT |
| | Forward primer | SEQ ID NO: 12 | CTCTGGTAACTAGAGATCCCTCAGACC-M | Reverse primer-extended |
| 118 | SEQ ID NO: 1 | SEQ ID NO: 23 | TGTGCTCATCTGTTCAACCCTG-O | SEQ ID NO: 20 |
| | TCAATAAAGCTTGCCTTGAG | SEQ ID NO: 4 | CCATCCATTCGGTAACTCTGGTA-N | CTGTTCGGGCGCCACT |
| | Forward primer | SEQ ID NO: 13 | ACTCTGGTAACTAGAGATCCCTCAGACC-M | Reverse primer-extended |
| 119 | SEQ ID NO: 1 | SEQ ID NO: 23 | TGTGCTCATCTGTTCAACCCTG-O | SEQ ID NO: 20 |
| | TCAATAAAGCTTGCCTTGAG | SEQ ID NO: 4 | CCATCCATTCGGTAACTCTGGTA-N | CTGTTCGGGCGCCACT |
| | Forward primer | SEQ ID NO: 14 | GACTCTGGTAACTAGAGATCCCTCAGACC-M | Reverse primer-extended |
| 120 | SEQ ID NO: 1 | SEQ ID NO: 23 | TGTGCTCATCTGTTCAACCCTG-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 8 | TCTGGTAACTAGAGATCCCTCAGAC-M | Reverse primer-extended |
| 121 | SEQ ID NO: 1 | SEQ ID NO: 23 | TGTGCTCATCTGTTCAACCCTG-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 9 | CTGGTAACTAGAGATCCCTCAGACC-M | Reverse primer-extended |
| 122 | SEQ ID NO: 1 | SEQ ID NO: 23 | TGTGCTCATCTGTTCAACCCTG-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 10 | CTGGTAACTAGAGATCCCTCAGAC-M | Reverse primer-extended |
| 123 | SEQ ID NO: 1 | SEQ ID NO: 23 | TGTGCTCATCTGTTCAACCCTG-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 11 | TGGTAACTAGAGATCCCTCAGACC-M | Reverse primer-extended |
| 124 | SEQ ID NO: 1 | SEQ ID NO: 23 | TGTGCTCATCTGTTCAACCCTG-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 12 | CTCTGGTAACTAGAGATCCCTCAGACC-M | Reverse primer-extended |
| 125 | SEQ ID NO: 1 | SEQ ID NO: 23 | TGTGCTCATCTGTTCAACCCTG-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 13 | ACTCTGGTAACTAGAGATCCCTCAGACC-M | Reverse primer-extended |
| 126 | SEQ ID NO: 1 | SEQ ID NO: 23 | TGTC3CTCATCTGTTCAACCCTG-O | SEQ ID NO: 20 |
| | TCAATAAAGCTTGCCTTGAG | SEQ ID NO: 6 | CCATCCATTCGGTAACTCTGGT-N | CTGTTCGGGCGCCACT |
| | Forward primer | SEQ ID NO: 14 | GACTCTGGTAACTAGAGATCCCTCAGACC-M | Reverse primer-extended |
| 127 | SEQ ID NO: 1 | SEQ ID NO: 5 | TGCTCATCTGTTCAACGCTGG-O | SEQ ID NO:21 |
| | TCAATAAAGCTTGCCTTGAG | SEQ ID NO: 4 | CCATCCATTCGGTAACTCTGGTA-N | CTGTTCGGGCGCCACTG |
| | Forward primer | SEQ ID NO: 8 | TCTGGTAACTAGAGATCCCTCAGAC-M | Reverse primer-extended |
| 128 | SEQ ID NO: 1 | SEQ ID NO: 5 | TGCTCATCTGTTCAACCCTGG-O | SEQ ID NO: 21 |
| | TCAATAAAGCTTGCCTTGAG | SEQ ID NO: 4 | CCATCCATTCGGTAACTCTGGTA-N | CTGTTCGGGCGCCACTG |
| | Forward primer | SEQ ID NO: 9 | CTGGTAACTAGAGATCCCTCAGACC-M | Reverse primer-extended |
| 129 | SEQ ID NO:1 | SEQ ID NO: 5 | TGCTCATCTGTTCAACCCTGG-O | SEQ ID NO: 21 |
| | TCAATAAAGCTTGCCTTGAG | SEQ ID NO: 4 | GCATGCATTCGGTAACTCTGGTA-N | CTGTTCGGGCGCCACTG |
| | Forward primer | SEQ ID NO: 10 | CTGGTAACTAGAGATCCCTCAGAC-M | Reverse primer-extended |
| 130 | SEQ ID NO: 1 | SEQ ID NO: 5 | TGCTCATCTGTTCAACCCTGG-O | SEQ ID NO: 21 |
| | TCAATAAAGCTTGCCTTGAG | SEQ ID NO: 4 | CCATCCATTCGGTAACTCTGGTA-N | CTGTTCGGGCGCCACTG |
| | Forward primer | SEQ ID NO: 11 | TGGTAACTAGAGATCCCTCAGACC-M | Reverse primer-extended |
| 131 | SEQ ID NO: 1 | SEQ ID NO: 5 | TGCTCATCTGTTCAACCCTGG-O | SEQ ID NO: 21 |
| | TCAATAAAGCTTGCCTTGAG | SEQ ID NO: 4 | CCATCCATTCGGTAACTCTGGTA-N | CTGTTCGGGCGCCACTG |
| | Forward primer | SEQ ID NO: 12 | CTCTGGTAACTAGAGATCCCTCAGACC-M | Reverse primer-extended |
| 132 | SEQ ID NO: 1 | SEQ ID NO: 5 | TGCTCATCTGTTCAACCCTGG-O | SEQ ID NO: 21 |
| | TCAATAAAGCTTGCCTTGAG | SEQ ID NO: 4 | CCATCCATTCGGTAACTCTGGTA-N | CTGTTCGGGCGCCACTG |
| | Forward primer | SEQ ID NO: 13 | ACTCTGGTAAGTAGAGATCCCTCAGACC-M | Reverse primer-extended |
| 133 | SEQ ID NO: 1 | SEQ ID NO: 5 | TGCTCATCTGTTGAACCCTGG-O | SEQ ID NO: 21 |
| | TCAATAAAGCTTGCCTTGAG | SEQ ID NO: 4 | CCATCCATTCGGTAACTCTGGTA-N | CTGTTCGGGCGCCACTG |
| | Forward primer | SEQ ID NO: 14 | GACTCTGGTAACTAGAGATCCCTCAGACC-M | Reverse primer-extended |
| 134 | SEQ ID NO: 1 | SEQ ID NO: 5 | TGCTCATCTGTTCAACCCTGG-O | SEQ ID NO: 21 |
| | TCAATAAAGCTTGCCTTGAG | SEQ ID NO: 6 | CCATGCATTCGGTAACTCTGGT-N | CTGTTCGGGCGCCACTG |
| | Forward primer | SEQ ID NO: 8 | TCTGGTAACTAGAGATCCCTCAGAC-M | Reverse primer-extended |
| 135 | SEQ ID NO: 1 | SEQ ID NO: 5 | TGCTCATCTGTTCAACCCTGG-O | SEQ ID NO: 21 |
| | TCAATAAAGCTTGCCTTGAG | SEQ ID NO: 6 | CCATCCATTCGGTAACTCTGGT-N | CTGTTCGGGCGCCACTG |
| | Forward primer | SEQ ID NO: 9 | CTGGTAACTAGAGATCCCTCAGACC-M | Reverse primer-extended |
| 136 | SEQ ID NO: 1 | SEQ ID NO: 5 | TGCTCATCTGTTCAACCCTGG-O | SEQ ID NO: 21 |
| | TCAATAAAGCTTGCCTTGAG | SEQ ID NO: 6 | CCATCCATTCGGTAACTCTGGT-N | CTGTTCGGGCGCCACTG |
| | Forward primer | SEQ ID NO: 10 | CTGGTAACTAGAGATCCCTCAGAC-M | Reverse primer-extended |
| 137 | SEQ ID NO: 1 | SEQ ID NO: 5 | TGCTCATCTGTTCAACCCTGG-O | SEQ ID NO: 21 |
| | TCAATAAAGCTTGCCTTGAG | SEQ ID NO: 6 | GCATCCATTCGGTAACTCTGGT-N | CTGTTCGGGCGCCACTG |
| | Forward primer | SEQ ID NO: 11 | TGGTAACTAGAGATCCCTCAGACC-M | Reverse primer-extended |
| 138 | SEQ ID NO: 1 | SEQ ID NO: 5 | TGCTCATCTGTTCAACCCTGG-O | SEQ ID NO: 21 |
| | TCAATAAAGCTTGCCTTGAG | SEQ ID NO: 6 | CCATCCATTCGGTAACTCTGGT-N | CTGTTCGGGCGCCACTG |
| | Forward primer | SEQ ID NO: 12 | CTCTGGTAACTAGAGATCCCTCAGACC-M | Reverse primer-extended |
| 139 | SEQ ID NO: 1 | SEQ ID NO: 5 | TGCTCATCTGTTCAACCCTGG-O | SEQ ID NO: 21 |
| | TCAATAAAGCTTGCCTTGAG | SEQ ID NO: 6 | CCATCCATTCGGTAACTCTGGT-N | CTGTTCGGGCGCCACTG |
| | Forward primer | SEQ ID NO: 13 | ACTCTGGTAACTAGAGATCCCTCAGACC-M | Reverse primer-extended |
| 140 | SEQ ID NO: 1 | SEQ ID NO: 5 | TGCTCATCTGTTCAACCCTGG-O | SEQ ID NO: 21 |
| | TCAATAAAGGTTGCCTTGAG | SEQ ID NO: 6 | CCATCCATrCGGTAACTCTGGT-N | CTGTTCGGGCGCCACTG |
| | Forward primer | SEQ ID NO: 14 | GACTCTGGTAACTAGAGATCCCTCAGACC-M | Reverse primer-extended |
| 141 | SEQ ID NO: 1 | SEQ ID NO: 7 | TGTGCTCATCTGTTCAACCCTGT-O | SEQ ID NO: 21 |
| | TCAATAAAGCTTGCCTTGAG | SEQ ID NO: 4 | CCATCCATTGGGTAACTCTGGTA-N | CTGTTCGGGCGCCACTG |
| | Forward primer | SEQ ID NO: 8 | TCTGGTAACTAGAGATCCCTCAGAC-M | Reverse primer-extended |
| 142 | SEQ ID NO: 1 | SEQ ID NO: 7 | TGTGCTCATCTGTTCAACCCTGT-O | SEQ ID NO: 21 |
| | TCAATAAAGCTTGCCTTGAG | SEQ ID NO: 4 | CCATCCATTCGGTAACTCTGGTA-N | CTGTTCGGGCGCCACTG |
| | Forward primer | SEQ ID NO: 9 | CTGGTAACTAGAGATCCCTCAGACC-M | Reverse primer-extended |
| 143 | SEQ ID NO: 1 | SEQ ID NO: 7 | TGTGCTCATCTGTTCAACCCTGT-O | SEQ ID NO: 21 |
| | TCAATAAAGCTTGCCTTGAG | SEQ ID NO: 4 | CCATCCATTGGGTAACTCTGGTA-N | CTGTGCGGGCGCCACTG |
| | Forward primer | SEQ ID NO: 10 | CTGGTAACTAGAGATCCCTCAGAC-M | Reverse primer-extended |
| 144 | SEQ ID NO: 1 | SEQ ID NO: 7 | TGTGCTCATCTGTTCAACCCTGT-O | SEQ ID NO: 21 |
| | TCAATAAAGCTTGCCTTGAG | SEQ ID NO: 4 | CCATCCATTGGGTAACTCTGGTA-N | CTGTTCGGGCGCCACTG |
| | Forward primer | SEQ ID NO: 11 | TGGTAACTAGAGATCCCTCAGACC-M | Reverse primer extended |
| 145 | SEQ ID NO: 1 | SEQ ID NO: 7 | TGTGCTCATCTGTTCAACCCTGT-O | SEQ ID NO: 21 |
| | TCAATAAAGCTTGCCTTGAG | SEQ ID NO: 4 | CCATCCATTCGGTAACTCTGGTA-N | CTGTTCGGGCGCCACTG |
| | Forward primer | SEQ ID NO: 12 | CTCTGGTAACTAGAGATCCCTCAGACC-M | Reverse primer-extended |
| 146 | SEQ ID NO: 1 | SEQ ID NO: 7 | TGTGCTCATCTGTTCAACCCTGT-O | SEQ ID NO: 21 |
| | TCAATAAAGCTTGCCTTGAG | SEQ ID NO: 4 | CCATCGATTCGGTAACTCTGGTA- N | CTGTTCGGGCGCCACTG |
| | Forward primer | SEQ ID NO: 13 | ACTCTGGTAACTAGAGATCCCTCAGACC-M | Reverse primer-extended |
| 147 | SEQ ID NO: 1 | SEQ ID NO: 7 | TGTGCTCATCTGTTCAACCCTGT-O | SEQ ID NO: 21 |
| | TCAATAAAGCTTGCCTTGAG | SEQ ID NO: 4 | CCATCCATTCGGTAACTCTGGTA-N | CTGTTCGGGCGCCACTG |
| | Forward primer | SEQ ID NO: 14 | GACTCTGGTAACTAGAGATCCCTCAGACC-M | Reverse primer-extended |
| 148 | SEQ ID NO: 1 | SEQ ID NO: 7 | TGTGCTCATCTGTTCAACCCTGT-O | SEQ ID NO: 21 |
| | TCAATAAAGCTTGCCTTGAG | SEQ ID NO: 6 | CCATCCATTCGGTAACTCTGGT-N | CTGTTCGGGCGCCACTG |
| | Forward primer | SEQ ID NO: 8 | TCTGGTAACTAGAGATCCCTCAGAC-M | Reverse primer-extended |
| 149 | SEQ ID NO: 1 | SEQ ID NO: 7 | TGTGCTCATCTGTTCAACCCTGT-O | SEQ ID NO: 21 |
| | TCAATAAAGCTTGCCTTGAG | SEQ ID NO: 6 | CCATCCATTCGGTAACTCTGGT-N | CTGTTCGGGCGCCACTG |
| | Forward primer | SEQ ID NO: 9 | CTGGTAACTAGAGATCCCTCAGACC-M | Reverse primer-extended |
| 150 | SEQ ID NO: 1 | SEQ ID NO: 7 | TGTGCTCATCTGTTCAACCCTGT-O | SEQ ID NO: 21 |
| | TCAATAAAGCTTGCCTTGAG | SEQ ID NO: 6 | CCATCCATTCGGTAACTCTGGT-N | CTGTTCGGGCGCCACTG |
| | Forward primer | SEQ ID NO: 10 | CTGGTAACTAGAGATCCCTCAGAC-M | Reverse primer-extended |
| 151 | SEQ ID NO: 1 | SEQ ID NO: 7 | TGTGCTCATCTGTTCAACCCTGT-O | SEQ ID NO: 21 |
| | TCAATAAAGCTTGCCTTGAG | SEQ ID NO: 6 | CCATCCATTCGGTAACTCTGGT-N | CTCTTCGGGCGCCACTG |
| | Forward primer | SEQ ID NO: 11 | TGGTAACTAGAGATCCCTCAGACC-M | Reverse primer-extended |
| 152 | SEQ ID NO: 1 | SEQ ID NO: 7 | TGTGCTCATCTGTTCAACCCTGT-O | SEQ ID NO: 21 |
| | TCAATAAAGCTTGCCTTGAG | SEQ ID NO: 6 | CCATCCATTCGGTAACTCTGGT-N | CTGTTCGGGCGCCACTG |
| | Forward primer | SEQ ID NO: 12 | CTCTGGTAACTAGAGATCCCTCAGACC-M | Reverse primer-extended |
| 153 | SEQ ID NO: 1 | SEQ ID NO: 7 | TGTGCTCATCTGTTCAACCCTGT-O | SEQ ID NO: 21 |
| | TCAATAAAGCTTGCCTTGAG | SEQ ID NO: 6 | CCATCCATTCGGTAACTCTGGT-N | CTGTTCGGGCGCCACTG |
| | Forward primer | SEQ ID NO: 13 | ACTCTGGTAACTAGAGATCCCTCAGACC-M | Reverse primer-extended |
| 154 | SEQ ID NO: 1 | SEQ ID NO:7 | TGTGCTCATCTGTTCAACCCTGT-O | SEQ ID NO: 21 |
| | TCAATAAAGCTTGCCTTGAG | SEQ ID NO: 6 | CCATCCATTCGGTAACTCTGGT-N | CTGTTCGGGCGCCACTG |
| | Forward primer | SEQ ID NO: 14 | GACTCTGGTAACTAGAGATCCCTCAGACC-M | Reverse primer-extended |
| 155 | SEQ ID NO: 1 | SEQ ID NO: 23 | TGTGCTCATCTGTTCAACCCTG-O | SEQ ID NO: 21 |
| | TCAATAAAGCTTGCCTTGAG | SEQ ID NO: 4 | CCATCCATTCGGTAACTCTGGTA-N | CTGTTCGGGCGCCACTG |
| | Forward primer | SEQ ID NO: 8 | TCTGGTAACTACIAGATCGCTCAGAC-M | Reverse primer-extended |
| 156 | SEQ ID NO: 1 | SEQ ID NO: 23 | TGTGCTCATCTGTTCAACCCTG-O | SEQ ID NO: 21 |
| | TCAATAAAGCTTGCCTTGAG | SEQ ID NO: 4 | CCATCCATCGGTAACTCTGGTA-N | CTGTTCGGGCGCCACTG |
| | Forward primer | SEQ ID NO: 9 | CTGGTAACTAGAGATCCCTCAGACC-M | Reverse primer-extended |
| 157 | SEQ ID NO: 1 | SEQ ID NO: 23 | TGTGCTCATCTGTTCAACCCTG-O | SEQ ID NO: 21 |
| | TCAATAAAGCTTGCCTTGAG | SEQ ID NO: 4 | CCATCCATTCGGTAACTCTGGTA-N | CTGTTCGGGCGCCACTG |
| | Forward primer | SEQ ID NO: 10 | CTGGTAACTAGAGATCCCTCAGAC-M | Reverse primer-extended |
| 158 | SEQ ID NO: 1 | SEQ ID NO: 23 | TGTGCTCATCTGTTCAACCCTG-O | SEQ ID NO :21 |
| | TCAATAAAGCTTGCCTTGAG | SEQ ID NO: 4 | CCATCCATTCGGTAACTCTGGTA-N | CTGTTCGGGCGCCACTG |
| | Forward primer | SEQ ID NO: 11 | TGGTAACTAGAGATCGCTCAGACC-M | Reverse primer-extended |
| 159 | SEQ ID NO: 1 | SEQ ID NO: 23 | TGTGCTCATCTGTTCAACCCTG-O | SEQ ID NO: 21 |
| | TCAATAAAGCTTGCCTTGAG | SEQ ID NO: 4 | CCATCCATTCGGTAACTCTGGTA-N | CTGTTCGGGCGCCACTG |
| | Forward primer | SEQ ID NO: 12 | CTCTGGTAACTAGAGATCCCTCAGACC-M | Reverse primer-extended |
| 160 | SEQ ID NO: 1 | SEQ ID NO: 23 | TGTGCTCATCTGTTCAACCCTG-O | SEQ ID NO:2 1 |
| | TCAATAAAGCTTGCCTTGAG | SEQ ID NO: 4 | CCATCCATTCGGTAACTCTGGTA-N | CTGTTCGGGCGCCACTG |
| | Forward primer | SEQ ID NO: 13 | ACTCTGGTAACTAGAGATCCCTCAGACC-M | Reverse primer-extended |
| 161 | SEQ ID NO: 1 | SEQ ID NO: 23 | TGTGCTCATCTGTTCAACCCTG-O | SEQ ID NO: 21 |
| | TCAATAAAGCTTGCCTTGAG | SEQ ID NO: 4 | CCATCCATTCGGTAACTCTGGTA- N | CTGTTCGGGCGCCACTG |
| | Forward primer | SEQ ID NO: 14 | GACTCTGGTAACTAGAGATCCCTCAGACC-M | Reverse primer-extended |
| 162 | SEQ ID NO: 1 | SEQ ID NO: 23 | TGTGCTCATCTGTTCAACCCTG-O | SEQ ID NO: 21 |
| | TCAATAAAGCTTGCCTTGAG | SEQ ID NO: 6 | CCATCCATTCGGTAACTCTGGT-N | CTGTTCGGGCGCCACTG |
| | Forward primer | SEQ ID NO: 8 | TCTGGTAACTAGAGATCCCTCAGAC-M | Reverse primer-extended |
| 163 | SEQ ID NO: 1 | SEQ ID NO: 23 | TGTGCTCATCTGTTCAACCCTG-O | SEQ ID NO: 21 |
| | TCAATAAAGCTTGCCTTGAG | SEQ ID NO: 6 | CCATCCATTCGGTAACTCTGGT-N | CTGTTCGGGCGCCACTG |
| | Forward primer | SEQ ID NO: 9 | CTGGTAACTAGAGATCCCTCAGACC-M | Reverse primer-extended |
| 164 | SEQ ID NO: 1 | SEQ ID NO: 23 | TGTGCTCATCTGTTCAACCCTG-O | SEQ ID NO: 21 |
| | TCAATAAAGCTTGCCTTGAG | SEQ ID NO: 6 | CCATCCATTCGGTAACTCTGGT-N | CTGTTCGGGCGCCACTG |
| | Forward primer | SEQ ID NO: 10 | CTGGTAACTAGAGATCCCTCAGAC-M | Reverse primer-extended |
| 165 | SEQ ID NO: 1 | SEQ ID NO: 23 | TGTGCTCATCTGTTCAACCCTG-O | SEQ ID NO: 21 |
| | TCAATAAAGCTTGCCTTGAG | SEQ ID NO: 6 | CCATCCATTCGGTAACTCTGGT-N | CTGTTCGGGCGCCACTG |
| | Forward primer | SEQ ID NO: 11 | TGGTAACTAGAGATCCCTCAGACC-M | Reverse primer-extended |
| 166 | SEQ ID NO: 1 | SEQ ID NO: 23 | TGTGCTCATCTGTTCAACCCTG-O | SEQ ID NO: 21 |
| | TCAATAAAGCTTGCCTTGAG | SEQ ID NO: 6 | CCATCCATTCGGTAACTCTGGT-N | CTGTTCGGGCGGCACTG |
| | Forward primer | SEQ ID NO: 12 | CTCTGGTAACTAGAGATCCGTCAGACC-M | Reverse primer-extended |
| 167 | SEQ ID NO: 1 | SEQ ID NO: 23 | TGTGCTCATCTGTTCAACCCTG-O | SEQ ID NO: 21 |
| | TCAATAAAGCTTGCCTTGAG | SEQ ID NO: 6 | CCATCCATTCGGTAACTCTGGT-N | CTGTTCGGGCGCCACTG |
| | Forward primer | SEQ ID NO: 13 | ACTCTGGTAACTAGAGATCCCTGAGACC-M | Reverse primer-extended |
| 168 | SEQ ID NO: 1 | SEQ ID NO: 23 | TGTGCTCATCTGTTCAACCCTG-O | SEQ ID NO: 21 |
| | TCAATAAAGCTTGCCTTGAG | SEQ ID NO: 6 | CCATCCATTCGGTAACTCTGGT-N | CTGTTCGGGCGCCACTG |
| | Forward primer | SEQ ID NO: 14 | GACTCTGGTAACTAGAGATCCCTCAGACC-M | Reverse primer-extended |
| 169 | SEQ ID NO: 1 | SEQ ID NO: 5 | TGCTCATCTGTTCAACCCTGG-O | SEQ ID NO: 22 |
| | TCAATAAAGCTTGCCTTGAG | SEQ ID NO: 4 | CCATCCATTCGGTAACTCTGGTA-N | CCTGTTCGGGCGCCACTG |
| | Forward primer | SEQ ID NO: 8 | TCTGOTAACTAGAGATCCCTCAGAC-M | C Reverse primer-extended |
| 170 | SEQ ID NO: 1 | SEQ ID NO: 5 | TGCTCATCTGTTCAACCCTGG-O | SEQ ID NO: 22 |
| | TCAATAAAGCTTGCCTTGAG | SEQ ID NO: 4 | CCATCCATTCGGTAACTCTGGTA-N | CCTGTTCGGGCGCCACTG |
| | Forward primer | SEQ ID NO: 9 | CTGGTAACTAGAGATCCCTCAGACC-M | C Reverse primer-extended |
| 171 | SEQ ID NO: 1 | SEQ ID NO: 5 | TGCTCATCTGTTCAACCCTGG-O | SEQ ID NO: 22 |
| | TCAATAAAGCTTGCCTTGAG | SEQ ID NO: 4 | CCATCCATTCGGTAACTCTGGTA-N | CCTGTTCGGGCGCCACTG |
| | Forward primer | SEQ ID NO: 10 | CTGGTAACTAGAGATCCCTCAGAC-M | C Reverse primer-extended |
| 172 | SEQ ID NO: 1 | SEQ ID NO: 5 | TGCTCATCTGTTCAACCCTGG-O | SEQ ID NO: 22 |
| | TCAATAAAGCTTGCCTTGAG | SEQ ID NO: 4 | CCATCCATTCGGTAACTCTGGTA-N | CCTGTTCGGGCGCCACTG |
| | Forward primer | SEQ ID NO: 11 | TGGTAACTAGAGATCCCTCAGACC-M | C Reverse primer-extended |
| 173 | SEQ ID NO: 1 | SEQ ID NO: 5 | TGCTCATCTGTTCAACCCTGG-O | SEQ ID NO: 22 |
| | TCAATAAAGCTTGCCTTGAG | SEQ ID NO: 4 | CCATCCATTCGGTAACTCTGGTA-N | CCTGTTCGGGCGCCACTG |
| | Forward primer | SEQ ID NO: 12 | CTCTGGTAACTAGAGATCCCTCAGACC-M | C Reverse primer-extended |
| 174 | SEQ ID NO: 1 | SEQ ID NO: 5 | TGCTCATCTGTTCAACCCTGG-O | SEQ ID NO: 22 |
| | TCAATAAAGCTTGCCTTGAG | SEQ ID NO: 4 | CCATCCATTCGGTAACTCTGGTA-N | CCTGTTCGGGCGCCACTG |
| | Forward primer | SEQ ID NO: 13 | ACTCTGGTAACTAGAGATCCCTCAGACC-M | C Reverse primer-extended |
| 175 | SEQ ID NO: 1 | SEQ ID NO: 5 | TGCTCATCTGTTCAACCCTGG-O | SEQ ID NO: 22 |
| | TCAATAAAGCTTGCCTTGAG | SEQ ID NO: 4 | CCATCCATTCGGTAACTCTGGTA-N | CCTGTTCGGGCGCCACTG |
| | Forward primer | SEQ ID NO: 14 | GACTCTGGTAACTAGAGATCCCTCAGACC-M | C Reverse primer-extended |
| 176 | SEQ ID NO: 1 | SEQ ID NO: 5 | TGCTCATCTGTTCAACCCTGG-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 8 | TCTGGTAACTAGAGATCCCTCAGAC-M | C Reverse primer-extended |
| 177 | SEQ ID NO: 1 | SEQ ID NO: 5 | TGCTCATCTGTTCAACCCTGG-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 9 | CTGGTAACTAGAGATCCCTCAGACC-M | C Reverse primer-extended |
| 178 | SEQ ID NO: 1 | SEQ ID NO: 5 | TGCTCATCTGTTCAACCCTGG-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 10 | CTGGTAACTAGAGATCCCTCAGAC-M | C Reverse primer-extended |
| 179 | SEQ ID NO: 1 | SEQ ID NO: 5 | TGCTCATCTGTTCAACCCTGG-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 11 | TGGTAACTAGAGATCCCTCAGACC-M | C Reverse primer-extended |
| 180 | SEQ ID NO: 1 | SEQ ID NO: 5 | TGCTCATCTGTTCAACCCTGG-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 12 | CTCTGGTAACTAGAGATCCCTCAGACC-M | C Reverse primer-extended |
| 181 | SEQ ID NO: 1 | SEQ ID NO: 5 | TGCTCATCTGTTCAACCCTGG-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 13 | ACTCTGGTAACTAGAGATCCCTCAGACC-M | C Reverse primer-extended |
| 182 | SEQ ID NO: 1 | SEQ ID NO: 5 | TGCTCATCTGTTCAACCCTGG-O | SEQ ID NO: 22 |
| | TCAATAAAGCTTGCCTTGAG | SEQ ID NO: 6 | CCATCCATTCGGTAACTCTGGT-N | CCTGTTCGGGCGCGACTG |
| | Forward primer | SEQ ID NO: 14 | GACTCTGGTAACTAGAGATCCCTCAGACC-M | C Reverse primer-extended |
| 183 | SEQ ID NO: 1 | SEQ ID NO: 7 | TGTGCTCATCTGTTCAACCCTCST-O | SEQ ID NO: 22 |
| | TCAATAAAGCTTGCCTTGAG | SEQ ID NO: 4 | CCATCCATTCGGTAACTCTGGTA-N | CCTGTTCGGGCGCCACTG |
| | Forward primer | SEQ ID NO: 8 | TCTGGTAACTAGAGATCCCTCAGAC-M | C Reverse primer-extended |
| 184 | SEQ ID NO: 1 | SEQ ID NO: 7 | TGTGCTCATCTGTTCAACCCTGT-O | SEQ ID NO: 22 |
| | TCAATAAAGCTTGCCTTGAG | SEQ ID NO: 4 | CCATCCATTCGGTAACTCTGGTA-N | CCTGTTCGOGCGCCACTG |
| | Forward primer | SEQ ID NO: 9 | CTGGTAACTAGAGATCCCTCAGACC-M | C Reverse primer-extended |
| 185 | SEQ ID NO: 1 | SEQ ID NO: 7 | TGTGCTCATCTGTTCAACCCTGT-O | SEQ ID NO: 22 |
| | TCAATAAAGCTTGCCTTGAG | SEQ ID NO: 4 | CCATCCATTCGGTAACTCTGGTA-N | CCTGTTCGGGCGCCACTG |
| | Forward primer | SEQ ID NO: 10 | CTGGTAACTAGAGATCCCTCAGAC-M | C Reverse primer-extended |
| 186 | SEQ ID NO: 1 | SEQ ID NO: 7 | TGTGCTCATCTGTTCAACCCTGT-O | SEQ ID NO: 22 |
| | TCAATAAAGCTTGCCTTGAG | SEQ ID NO: 4 | CCATCCATTCGGTAACTCTGGTA-N | CCTGTTCGGGCGCCACTG |
| | Forward primer | SEQ ID NO: 11 | TGC3TAACTAGAGATCCCTCAGACC-M | C Reverse primer-extended |
| 187 | SEQ ID NO: 1 | SEQ ID NO: 7 | TGTGCTCATCTGTTCAACCCTGT-O | SEQ ID NO: 22 |
| | TCAATAAAGCTTGCCTTGAG | SEQ ID NO: 4 | CCATCCATTCGGTAACTCTGGTA-N | CCTGTTCGGGCGCCACTG |
| | Forward primer | SEQ ID NO: 12 | CTCTGGTAACTAGAGATCCCTCAGACC-M | C Reverse primer-extended |
| 188 | SEQ ID NO: 1 | SEQ ID NO: 7 | TGTGCTCATCTGTTCAACCCTGT-O | SEQ ID NO: 22 |
| | TCAATAAAGCTTGCCTTGAG | SEQ ID NO: 4 | CCATCCATTCGGTAACTCTGGTA-N | CCTGTTCGGGCGCCACTG |
| | Forward primer | SEQ ID NO: 13 | ACTCTGGTAACTACTAGATCCCTCAGACC-M | C Reverse primer-extended |
| 189 | SEQ ID NO: 1 | SEQ ID NO: 7 | TGTGCTCATCTGTTCAACCCTGT-O | SEQ ID NO: 22 |
| | TCAATAAAGCTTGCCTTGAG | SEQ ID NO: 4 | CCATCCATTCGGTAACTCTGGTA-N | CCTGTTCGGGCGCCACTG |
| | Forward primer | SEQ ID NO: 14 | GACTCTGGTAACTAGAGATCCCTCAGACC-M | C Reverse primer-extended |
| 190 | SEQ ID NO: 1 | SEQ ID NO: 7 | TGTGCTCATCTGTTCAACCCTGT-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 8 | TCTGGTAACTAGAGATCCCTCAGAC-M | C Reverse primer-extended |
| 191 | SEQ ID NO: 1 | SEQ ID NO: 7 | TGTGCTCATCTGTTCAACCCTGT-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 9 | CTGGTAACTAGAGATCCCTCAGACC-M | C Reverse primer-extended |
| 192 | SEQ ID NO: 1 | SEQ ID NO: 7 | TGTGCTCATCTGTTCAACCCTGT-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 10 | CTGGTAACTAGAGATCCCTCAGAC-M | C Reverse primer-extended |
| 193 | SEQ ID NO: 1 | SEQ ID NO: 7 | TGTGCTCATCTGTTCAACCCTGT-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 11 | TGC3TAACTAGAGATCCCTCAGACC-M | C Reverse primer-extended |
| 194 | SEQ ID NO: 1 | SEQ ID NO: 7 | TGTGCTCATCTGTTCAACCCTGT-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 12 | CTCTGGTAACTAGAGATCCCTCAGACC-M | C Reverse primer-extended |
| 195 | SEQ ID NO: 1 | SEQ ID NO: 7 | TGTGCTCATCTGTTCAACCCTGT-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 13 | ACTCTGGTAACTAGACTATCCCTCAGACC-M | C Reverse primer-extended |
| 196 | SEQ ID NO: 1 | SEQ ID NO: 7 | TGTGCTCATCTGTTCAACCCTGT-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 14 | GACTCTGGTAACTAGAGATCCCTCAGACC-M | C Reverse primer-extended |
| 197 | SEQ ID NO: 1 | SEQ ID NO: 23 | TGTGCTCATCTGTTCAACCCTG-O | SEQ ID NO: 22 |
| | TCAATAAAGCTTGCCTTGAG | SEQ ID NO: 4 | CCATCCATTCGGTAACTCTGGTA-N | CCTGTTCGGGCGCCACTG |
| | Forward primer | SEQ ID NO: 8 | TCTGGTAACTAGAGATCCCTCAGAC-M | C Reverse primer-extended |
| 198 | SEQ ID NO: 1 | SEQ ID NO: 23 | TGTGCTCATCTCTTCAACCCTG-O | SEQ ID NO: 22 |
| | TCAATAAAGCTTGCCTTGAG | SEQ ID NO: 4 | CCATCCATTCGGTAACTCTOGTA-N | CCTGTTCGGGCGCCACTG |
| | Forward primer | SEQ ID NO: 9 | CTGGTAACTAGAGATCCCTCAGACC-M | C Reverse primer-extended |
| 199 | SEQ ID NO: 1 | SEQ ID NO: 23 | TGTGCTCATCTCTTCAACCCTG-O | SEQ ID NO: 22 |
| | TCAATAAAGCTTGCCTTGAG | SEQ ID NO: 4 | CCATCCATTCGGTAACTCTGGTA-N | CCTGTTCGGGCGCCACTG |
| | Forward primer | SEQ ID NO: 10 | CTGGTAACTAGAGATCCCTCAGAC-M | C Reverse primer-extended |
| 200 | SEQ ID NO: 1 | SEQ ID NO: 23 | TGTGCTCATCTGTTCAACCCTG-O | SEQ ID NO: 22 |
| | TCAATAAAGCTTGCCTTGAG | SEQ ID NO: 4 | CCATCCA1TCGGTAACTCTGGTA-N | CCTGTTCGGGCGCCACTG |
| | Forward primer | SEQ ID NO: 11 | TGGTAACTAGAGATCCCTCAGACC-M | C Reverse primer-extended |
| 201 | SEQ ID NO: 1 | SEQ ID NO: 23 | TGTGCTCATCTGTTCAACCCTG-O | SEQ ID NO: 22 |
| | TCAATAAAGCTTGCCTTGAG | SEQ ID NO: 4 | CCATCCATTCGGTAACTCTGGTA-N | CCTGTTCGGGCGCCACTG |
| | Forward primer | SEQ ID NO: 12 | CTCTGGTAACTAGAGATCCCTCAGACC-M | C Reverse primer-extended |
| 202 | SEQ ID NO: 1 | SEQ ID NO: 23 | TGTOCTCATCTGTTCAACCCTG-O | SEQ ID NO: 22 |
| | TCAATAAAGCTTGCCTTGAG | SEQ ID NO: 4 | CCATCCA1TCGGTAACTCTGGTA-N | CCTGTTCGGGCGCCACTG |
| | Forward primer | SEQ ID NO: 13 | ACTCTGCTAACTAGAGATCCCTCAGACC-M | C Reverse primer extended |
| 203 | SEQ ID NO: 1 | SEQ ID NO: 23 | TGTGCTCATCTGTTCAACCCTG-O | SEQ ID NO: 22 |
| | TCAATAAAGCTTGCCTTGAG | SEQ ID NO: 4 | CCATCCATTCGGTAACTCTGGTA-N | CCTGTTCGGGCGCCACTG |
| | Forward primer | SEQ ID NO: 14 | GACTCTGGTAACTAGAGATCCCTCAGACC-M | C Reverse primer-extended |
| 204 | SEQ ID NO: 1 | SEQ ID NO: 23 | TGTGCTCATCTGTTCAACCCTG-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 8 | TCTGGTAACTAGAGATCCCTCAGAC-M | C Reverse primer-extended |
| 205 | SEQ ID NO: 1 | SEQ ID NO: 23 | TGTGCTCATCTGTTCAACCCTG-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 9 | CTGGTAACTAGAGATCCCTCAGACC-M | C Reverse primer-extended |
| 206 | SEQ ID NO: 1 | SEQ ID NO: 23 | TGTGCTCATCTGTTCAACCCTG-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 10 | CTGGTAACTAGAGATCCCTCAGAC-M | C Reverse primer-extended |
| 207 | SEQ ID NO: 1 | SEQ ID NO: 23 | TGTGCTCATCTGTTCAACCCTG-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 11 | TGGTAACTAGAGATCCCTCAGACC-M | C Reverse primer-extended |
| 208 | SEQ ID NO: 1 | SEQ ID NO: 23 | TGTGCTCATCTGTTCAACCCTG-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 12 | CTCTGGTAACTACTAGATCCCTCAGACC-M | C Reverse primer-extended |
| 209 | SEQ ID NO: 1 | SEQ ID NO: 23 | TGTGCTCATCTGTTCAACCCTG-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 13 | ACTCTGGTAACTAGAGATCCCTCAGACC-M | C Reverse primer-extended |
| 210 | SEQ ID NO: 1 | SEQ ID NO: 23 | TGTGCTCATCTGTTCAACCCTG-O | SEQ ID NO: 22 |
| | TCAATAAAGCTTGCCTTGAG | SEQ ID NO: 6 | CCATCCATTCGGTAACTCTGGT-N | CCTGTTCGGGCGCCACTG |
| | Forward primer | SEQ ID NO: 14 | GACTCTGGTAACTAGAGATCCCTCAGAGC-M | C Reverse primer-extended |

### Other Embodiments

Other embodiments will be evident to those of skill in the art. It should be understood that the foregoing detailed description is provided for clarity only and is merely exemplary. The spirit and scope of the present invention are not limited to the above examples, but are encompassed by the following claims.

What is claimed is:

1. An isolated nucleic acid sequence comprising a sequence selected from the group consisting of: SEQ ID NOS:1-23.

2. A method of hybridizing the isolated nucleic acid of claim 1 to an isolated HIV-1 sequence or an HIV-1 sequence derived from an artificial construct comprising contacting the nucleic acid of claim 1 to a sample under conditions suitable for hybridization.

3. The method of claim 2, further comprising isolating the hybridized HIV-1 sequence.

4. The method of claim 2, further comprising sequencing the HIV-1 sequence.

5. A method of producing a nucleic acid product, comprising contacting one or more nucleic acid sequences of claim 1 with an HIV-1 template in the presence of a nucleic acid polymerase under conditions suitable for nucleic acid polymerization.

6. The method of claim 5, wherein the nucleic acid polymerase is a reverse transcriptase, and the one or more nucleic acid sequences are selected from the group consisting of: SEQ ID NOS:2 and 19-22.

7. The method of claim 5, wherein the nucleic acid product is a polymerase chain reaction amplicon produced using at least one forward primer selected from the group consisting of SEQ ID NOS:1, 3 and 15-18 and at least one reverse primer selected from the group consisting of SEQ ID NOS:2 and 19-22.

8. A primer pair for producing the nucleic acid of claim 7.

9. The primer pair of claim 8, wherein the primer pair is selected from the group consisting of sets 1-210 of Table 1.

10. A probe that hybridizes to the nucleic acid product of claim 5.

11. The probe of claim 10, wherein the probe comprises a sequence selected from the group consisting of: SEQ ID NOS:4-14 and 23.

12. The probe of claim 10, wherein the probe is labeled with a detectable label selected from the group consisting of: a fluorescent label, a chemiluminescent label, a quencher, a radioactive label, biotin and gold.

13. A set of probes that hybridize to the PCR amplicon of claim 7, wherein a first probe comprises the sequence of SEQ ID NO:4 or SEQ ID NO:6, a second probe comprises the sequence of SEQ ID NOS:5, 7 or 23, and a third probe comprises a sequence selected from the group consisting of: SEQ ID NOS:8-14.

14. The set of probes of claim 13, wherein the first probe is labeled with a first detectable label, the second probe is labeled with a second detectable label, and the third probe is labeled with a third detectable label.

15. The set of probes of claim 14, wherein the first, second and third detectable labels are selected from the group consisting of: a fluorescent label, a chemiluminescent label, a quencher, a radioactive label, biotin and gold.

16. A method for detecting, quantitating and grouping HIV-1 DNA in a sample, comprising:
a) contacting the HIV-1 DNA with at least one forward primer comprising the sequence selected from the group consisting of: SEQ ID NOS:1, 3 and 15-18 and at least one reverse primer comprising the sequence selected from the group consisting of: SEQ ID NOS:2 and 19-22 under conditions such that nucleic acid amplification occurs to yield a PCR amplicon;
b) contacting the PCR amplicon with one or more probes comprising one or more sequences selected from the group consisting of: SEQ ID NOS:4-14 and 23 under conditions such that hybridization or binding occurs;
wherein the hybridization or binding of the probe allows for detection, quantification and grouping of HIV-1 DNA in the sample.

17. The method of claim 16, wherein the HIV-1 DNA is a cDNA produced by contacting an HIV-1 RNA with a reverse transcriptase primer sequence selected from the group consisting of: SEQ ID NOS:2 and 19-22 under conditions suitable for the production of cDNA.

18. The method of claim 16, wherein each of the one or more probes is indicative of a group of HIV-1.

19. The method of claim 18, wherein each of the one or more probes is labeled with different fluorescent labels.

20. The method of claim 16, wherein the sample is selected from the group consisting of: blood, serum, plasma, sputum, urine, stool, skin, cerebrospinal fluid, saliva, gastric secretions, semen, seminal fluid, breastmilk, tears, oropharyngeal swabs, nasopharyngeal swabs, throat swabs, nasal aspirates, nasal wash, fluids collected from the ear, eye, mouth, respiratory airways, spinal tissue or fluid, cerebral fluid, trigeminal ganglion sample, a sacral ganglion sample, adipose tissue, lymphoid tissue, placental tissue, upper reproductive tract tissue, gastrointestinal tract tissue, male genital tissue and fetal central nervous system tissue.

21. The method of claim 20, wherein the sample is from a human.

22. The method of claim 16, wherein the at least one forward primer, the at least one reverse primer and the one or more probes is selected from the group consisting of: sets 1-210 of Table 1.

23. The method of claim 16 wherein the detecting, quantitating and grouping of HIV-1 DNA results in substantially no cross-reactivity with a sequence from an infectious agent selected from the group consisting of: Adenovirus types 2, 3 and 7; BK human polyoma virus; CMV; EBV; HHV-6 A; HHV-6 B; HHV-7; HHV-8; HIV-2; HSV-2; HSV-2; HTLV-I; HTLV-II; human hepatitis A virus; human hepatitis B virus; hepatitis C virus; JC virus; human papillomavirus types 16; human papillomavirus types 18; VZV; Candida albicans; Escherichia coli; Klebsiella pneumoniae; Morganella morganii; Proteus mirabilis; Pseudomonas aeruginosa; Serratia marcescens; Staphylococcus aureus; Staphylococcus haemolyticus; Staphylococcus hominis; Staphylococcus sciuri; Staphylococcus simulans; Staphylococcus epidermidis; and Staphylococcus intermedius.

24. A kit for detecting, quantitating and grouping an HIV-1 DNA sample, comprising:
a) at least one forward primer comprising the sequence selected from the group consisting of: SEQ ID NOS:1, 3 and 15-18;
b) at least one reverse primer comprising the sequence selected from the group consisting of: SEQ ID NO:2, and 19-22; and
c) one or more probes comprising a sequence selected from the group consisting of: SEQ ID NOS:4-14 and 23.

25. The kit of claim 24, further comprising a reverse transcriptase primer sequence selected from the group consisting of: SEQ ID NOS:2 and 19-22.

26. The kit of claim 24, wherein the one or more probes is indicative of a group of HIV-1.

27. The kit of claim 26, wherein the one or more probes are labeled with different fluorescent labels.

28. The kit of claim 24, wherein the at least one forward primer, the at least one reverse primer and the one or more probes are selected from the group consisting of: sets 1-210 of Table 1.

29. A method for diagnosing an HIV-1-associated condition or disease, comprising:
a) contacting a sample with at least one forward and reverse primer pair selected from the group consisting of: SEQ ID NOS:1-3 and 15-22;
b) conducting a reverse transcriptase-polymerase chain reaction, a polymerase chain reaction or signal amplification; and
c) detecting and grouping the generation of an amplified product using one or more probes selected from the group consisting of: SEQ ID NOS:4-14 and 23;
wherein the generation of an amplified product indicates the presence of HIV-1 virus in the sample.

30. The method of claim 29, wherein the sample is selected from the group consisting of: blood, serum, plasma, sputum, urine, stool, skin, cerebrospinal fluid, saliva, gastric secretions, semen, seminal fluid, breastmilk, tears, oropharyngeal swabs, nasopharyngeal swabs, throat swabs, nasal aspirates, nasal wash, fluids collected from the ear, eye, mouth, respiratory airways, spinal tissue or fluid, cerebral fluid, trigeminal ganglion sample, a sacral ganglion sample, adipose tissue, lymphoid tissue, placental tissue, upper reproductive tract tissue, gastrointestinal tract tissue, male genital tissue and fetal central nervous system tissue.

31. The method of claim 29, wherein the HIV-1-associated condition or disease is selected from the group consisting of: AIDS, Kaposi's sarcoma, Non-Hodgkins lymphoma, Pneumocystis carinii pneumonia, Pneumocystis jirovecii pneumonia, Candida esophagitis, Candida albicans infection, Pseudomonas aeruginosa infection, Staphylococcus aureus infection, Streptococcus pyogenes infection, Acinetobacter baumanni infection, Toxoplasma gondii infection, Toxoplasma encephalitis, Aspergillus infection, cryptosporidiosis, microsporidiosis, Cryptococcus neoformans infection, mycobacterium avium complex disseminated infection, Epstein-Barr virus infection, cytomegalovirus retinitis, progressive multifocal leukoencephalopathy from JC virus infection, HIV-associated dementia, central nervous system (CNS) malignancies, oral candidiasis, aseptic meningoencephalitis, disorders of the digestive tract, endocrine dysfunction, metabolic disorders, wasting syndrome, anemia, neutropenia, rheumatological syndromes, cervical cancer, anal cancer, rectal cancer, Burkitt's lymphoma, penicilliosis, tuberculosis, herpes virus 8 infection, herpes virus simplex 1 infection, human papillomavirus infection, cytomegalovirus infection, mycobacterial infection, rotavirus infection, adenovirus infection, astrovirus infection, esophagitis, chronic diarrhea due to Salmonella, Shigella, Listeria, or Campylobacter, and nephropathy.


SEQUENCE LISTING (23 sequences)

SEQ ID NO: 1 - DNA, 20 bp, Artificial Sequence
  Description of Artificial Sequence Synthetic primer
tcaataaagcttgccttgag

SEQ ID NO: 2 - DNA, 15 bp, Artificial Sequence
  Description of Artificial Sequence Synthetic primer
tgttcgggcgccact

SEQ ID NO: 3 - DNA, 19 bp, Artificial Sequence
  Description of Artificial Sequence Synthetic primer
caataaagcttgccttgag

SEQ ID NO: 4 - DNA, 23 bp, Artificial Sequence
  Description of Artificial Sequence Synthetic probe
ccatccattcggtaactctggta

SEQ ID NO: 5 - DNA, 21 bp, Artificial Sequence
  Description of Artificial Sequence Synthetic probe
tgctcatctgttcaaccctgg

SEQ ID NO: 6 - DNA, 22 bp, Artificial Sequence
  Description of Artificial Sequence Synthetic probe
ccatccattcggtaactctggt

SEQ ID NO: 7 - DNA, 23 bp, Artificial Sequence
  Description of Artificial Sequence Synthetic probe
tgtgctcatctgttcaaccctgt

SEQ ID NO: 8 - DNA, 25 bp, Artificial Sequence
  Description of Artificial Sequence Synthetic probe
tctggtaactagagatccctcagac

SEQ ID NO: 9 - DNA, 25 bp, Artificial Sequence
  Description of Artificial Sequence Synthetic probe
ctggtaactagagatccctcagacc

SEQ ID NO: 10 - DNA, 24 bp, Artificial Sequence
  Description of Artificial Sequence Synthetic probe
ctggtaactagagatccctcagac

SEQ ID NO: 11 - DNA, 24 bp, Artificial Sequence
  Description of Artificial Sequence Synthetic probe
tggtaactagagatccctcagacc

SEQ ID NO: 12 - DNA, 27 bp, Artificial Sequence
  Description of Artificial Sequence Synthetic probe
ctctggtaactagagatccctcagacc

SEQ ID NO: 13 - DNA, 28 bp, Artificial Sequence
  Description of Artificial Sequence Synthetic probe
actctggtaactagagatccctcagacc

SEQ ID NO: 14 - DNA, 29 bp, Artificial Sequence
  Description of Artificial Sequence Synthetic probe
gactctggtaactagagatccctcagacc

SEQ ID NO: 15 - DNA, 21 bp, Artificial Sequence
  Description of Artificial Sequence Synthetic primer
ctcaataaagcttgccttgag

SEQ ID NO: 16 - DNA, 21 bp, Artificial Sequence
  Description of Artificial Sequence Synthetic primer
tcaataaagcttgccttgagt

SEQ ID NO: 17 - DNA, 22 bp, Artificial Sequence
  Description of Artificial Sequence Synthetic primer
ctcaataaagcttgccttgagt

SEQ ID NO: 18 - DNA, 24 bp, Artificial Sequence
  Description of Artificial Sequence Synthetic primer
cctcaataaagcttgccttgagtg

SEQ ID NO: 19 - DNA, 16 bp, Artificial Sequence
  Description of Artificial Sequence Synthetic primer
tgttcgggcgccactg

SEQ ID NO: 20 - DNA, 16 bp, Artificial Sequence
  Description of Artificial Sequence Synthetic primer
ctgttcgggcgccact

SEQ ID NO: 21 - DNA, 17 bp, Artificial Sequence
  Description of Artificial Sequence Synthetic primer
ctgttcgggcgccactg

SEQ ID NO: 22 - DNA, 19 bp, Artificial Sequence
  Description of Artificial Sequence Synthetic primer
cctgttcgggcgccactgc

SEQ ID NO: 23 - DNA, 22 bp, Artificial Sequence
  Description of Artificial Sequence Synthetic probe
tgtgctcatctgttcaaccctg